SEQUENCE LISTING (15 sequences)

SEQ ID NO: 1 - DNA, 42 bp, Artificial
  Synthetic sequence
taatacgactcactatagggagaagccacatcgctcagacac

SEQ ID NO: 2 - DNA, 42 bp, Artificial
  Synthetic sequence
aattaaccctcactaaagggagactccatggtggtgaagacg

SEQ ID NO: 3 - DNA, 45 bp, Artificial
  Synthetic Sequence
taatacgactcactatagggagactgggatgttaaccagaagacc

SEQ ID NO: 4 - DNA, 44 bp, Artificial
  Synthetic sequence
aattaaccctcactaaagggagaagctggagtctggtctcatca

SEQ ID NO: 5 - DNA, 43 bp, Artificial
  Synthetic sequence
taatacgactcactatagggagattttgcctcctaggcatcac

SEQ ID NO: 6 - DNA, 43 bp, Artificial
  Synthetic sequence
aattaaccctcactaaagggagatgtggggctaggagattctg

SEQ ID NO: 7 - DNA, 30 bp, Artificial
  Synthetic Sequence
agcggataacagtcctgacgagacggaaga

SEQ ID NO: 8 - DNA, 31 bp, Artificial
  Synthetic Sequence
agcggataaccgtccaggagcttgtgaatta

SEQ ID NO: 9 - DNA, 29 bp, Artificial
  Synthetic Sequence
taggttgacaagctctcgactgcttgtgc

SEQ ID NO: 10 - DNA, 29 bp, Artificial
  Synthetic sequence
taggttgacctctccttctcgtgctgctt

SEQ ID NO: 11 - DNA, 16 bp, Artificial
  Synthetic sequence
atggggaaggtgaagg

SEQ ID NO: 12 - DNA, 17 bp, Artificial
  Synthetic sequence
cattgagcctcatgctc

SEQ ID NO: 13 - DNA, 21 bp, Artificial
  Synthetic sequence
tacaacaaatacccagatgct

SEQ ID NO: 14 - DNA, 18 bp, Artificial
  Synthetic sequence
aaggcctacactgctgtg

SEQ ID NO: 15 - DNA, 20 bp, Artificial
  Synthetic sequence
cccaggtcagatgtcatgta

(19) United States
(12) Patent Application Publication
(10) Pub. No.: US 2004/0009495 A1
(43) Pub. Date: Jan. 15, 2004

(54) Methods and products related to drug screening using gene expression patterns

(75) Inventors: Shawn O'Malley, Horseheads, NY (US); Ken Ross, Boston, MA (US); Kim Stegmaier, Brookline, MA (US); Todd Golub, Newton, MA (US); Brent Stockwell, Boston, MA (US)

Correspondence Address: WOLF GREENFIELD & SACKS, PC, FEDERAL RESERVE PLAZA, 600 ATLANTIC AVENUE, BOSTON, MA, 02210-2211, US

(73) Assignees: Whitehead Institute for Biomedical Research, Cambridge, MA; Dana-Farber Cancer Institute, Boston, MA

(21) Appl. No.: 10/313,335

(22) Filed: Dec. 6, 2002

Related U.S. Application Data
(60) Provisional application No. 60/341,005, filed on Dec. 7, 2001.

(51) Int. Cl. C12Q 1/68; G06F 19/00; G01N 33/48; G01N 33/50; C12P 19/34

(52) U.S. Cl. 435/6; 435/91.2; 702/20

(57) ABSTRACT

The invention involves high throughput methods for identifying properties of cells under a variety of cellular conditions. The high throughput methods have a variety of uses, including methods for identifying cellular modulators such as pharmacological agents or environmental conditions, methods for identifying a cellular phenotype and methods for identifying novel genes.

### RELATED APPLICATIONS

This application claims the benefit of priority of U.S. Provisional Application Serial No. 60/341,005, filed Dec. 7, 2001 which is incorporated by reference in its entirety.

### GOVERNMENT SUPPORT

Some aspects of the invention were made with government support under NIH training grant No. 5T3209172-27. The government may have certain rights in the invention.

### FIELD OF THE INVENTION

The invention relates in some aspects to high throughput methods for identifying properties of cells under a variety of cellular conditions. The methods are useful, for example, for identifying modulators such as pharmacological agents or environmental conditions that influence cellular properties.

### BACKGROUND OF THE INVENTION

High-density DNA microarrays, such as those commercially available from Affymetrix, Inc. (Santa Clara, Calif.), enable rapid and simultaneous quantitation of cellular mRNA levels. These cellular mRNA levels are indicative of the genes expressed in the cell. The gene expression profiles often comprise many genes and, thus, represent a unique signature of a physiological state of the cell or a cellular phenotype. A gene expression signature may be obtained in response to external stimuli such as temperature or ion changes, a drug and, a time course of drug. These gene expression signatures have also been shown to have utility as a diagnostic in predicting disease outcome or in detecting loss of heterogeneity.

### SUMMARY OF THE INVENTION

High throughput methods for screening compounds and identifying properties of cells have been discovered according to the invention. The methods utilize gene expression signatures and subsets thereof to predict cellular properties, which can then be used to identify the effects of multiple chemical compounds on cellular phenotype, to predict the status of a particular cell or to identify novel properties of a cell or cellular component such as a gene.

One aspect of the invention is a method of determining a gene expression profile for a cellular phenotype by establishing two or more sets of gene expression profiles, defining a set of marker genes that defines the differences between the two or more sets of gene expression profiles, and recording the set of marker genes in a database that defines the cellular phenotype.

This method may be used to determine the gene expression profile for many different cellular phenotypes. In some embodiments the cellular phenotype is of a cancer cell, a metastatic cancer cell, a cell resistant to radiation, a cell resistant to chemotherapy, a cancer cell that releases angiogenic factors, a cell with a positive drug response, a neutrophil, or a monocyte.

The gene expression profiles for the many possible cellular phenotypes may be determined for a variety of cell populations. In one embodiment the cell population is a cultured cell line. In another embodiment the cell population is an in vivo cell population. In a further embodiment the cell population is a population of cells from human peripheral blood.

Another aspect of the invention provides a method of screening a cell population. This method of screening is accomplished by defining a set of marker genes that represents a cellular phenotype, amplifying the set of marker genes from the cell population, determining the expression of the marker genes present in the cell population, and scoring the expression of the marker genes to screen the cell population for the cellular phenotype. In one embodiment the methods of the present invention may also be utilized to identify “metagenes”. In another embodiment the methods of the invention are used to define one or more “metagenes” in response to one or more drugs.

The cell population may be screened in response to a variety of external stimuli. In one embodiment the cell population is screened in response to a chemical compound. In another embodiment the chemical compound is selected from the group consisting of small molecule libraries, FDA approved drugs and synthetic chemical libraries.

The cell populations are screened in response to the external stimuli which may produce a cellular phenotype. In some embodiments the cellular phenotype is of a cancer cell, a metastatic cancer cell, a cell resistant to radiation, a cell resistant to chemotherapy, a cancer cell that releases angiogenic factors, a cell with a positive drug response, a neutrophil, or a monocyte.

To determine the cellular phenotype expressed by the cell population, a number of methods may be used to score the expression of the marker genes. In one embodiment the marker genes are scored relative to each other. In another embodiment the marker genes are scored on a binary basis. In still another embodiment the marker genes are scored relative to the expression of a control gene. In one embodiment the control gene is GAPDH. In another embodiment one or more of the marker genes is selected from the group consisting of IL1RN and SPP1. In still another embodiment of the invention one or more of the marker genes is selected from the group consisting of ORM1 and NCF1.

The set of marker genes of this invention may be used to define many cellular characteristics. In one embodiment the set of marker genes defines a set of phenotypic markers. In another embodiment the set of marker genes defines a set of therapeutic markers. In yet another embodiment the set of marker genes defines a set of diagnostic markers.

In addition to phenotype, the methods of the invention can be employed to define other biological characteristics of the cell population. In one embodiment the set of marker genes defines 1 or more novel genes. In another embodiment the set of marker genes represents a biological pathway. In yet another embodiment the set of marker genes defines a transcriptome.

The methods of this invention may also be used on a number of different types of cell populations from various sources. In one embodiment the cell population is a cultured cell line. In another embodiment the cell population is an HL60 cell line. In yet another embodiment the cell population is an in vivo cell population. In a further embodiment the cell population is a population of cells from human peripheral blood.

Yet another aspect of the invention provides a method for identifying an active compound. This is accomplished by contacting cells with a plurality of chemical compounds, amplifying a set of marker genes from the cells to determine the expression of marker genes present in the cells, and scoring the expression of the marker genes to identify a cellular phenotype, the presence of a specific cellular phenotype being indicative of an active compound. In one embodiment the plurality of chemical compounds is a set of compounds selected from the group consisting of small molecule libraries, FDA approved drugs, synthetic chemical libraries, phage display libraries, dosage libraries. In another embodiment the active compound is an anti-cancer drug. In a further embodiment the active compound is a cellular differentiation factor.

In another embodiment of the method, the set of marker genes which identified the cellular phenotype is a metagene. Marker genes and/or metagenes may be used to describe many phenotypes. In one embodiment the cellular phenotype is a tumorigenic status of the cell. In another embodiment the cellular phenotype is a metastatic status of the cell. In a yet another embodiment the set of marker genes is a cancer versus non-cancer marker gene set. In a further embodiment the set of marker genes is a metastatic versus non-metastatic marker gene set. In still another embodiment the set of marker genes is a radiation resistant versus radiation sensitive marker gene set. In yet another embodiment the set of marker genes is a chemotherapy resistant versus chemotherapy sensitive marker gene set. In another embodiment the cellular phenotype is a cellular differentiation status.

In order to determine the phenotype of the cell population, the expression of the marker genes and/or metagenes must be determined. One of ordinary skill in the art would appreciate the number of ways that are available to determine this expression. In one embodiment the expression of the marker genes is determined by custom reverse microarray analysis. In another embodiment the expression of the marker genes is determined by mass spectrometry.

Another aspect of the invention provides a method for identifying a cellular phenotype. This method of the invention is conducted by identifying the expression of metagenes in a cell to identify a cellular phenotype of the cell. In one embodiment the expression of metagenes is identified by amplifying signature genes characteristic of the metagenes from the cells. In another embodiment the cellular phenotype is identified by scoring the expression of the metagenes on a binary basis.

There also are a number of cellular phenotypes that may be identified with this method. In one embodiment the cellular phenotype is a cellular differentiation status. In another embodiment the cellular phenotype is a tumorigenic status of the cell.

Another aspect of the invention is a method for identifying a function of a gene by contacting cells with a diverse array of chemical compounds, amplifying a set of marker genes characteristic of a transcriptome from the cells to determine the expression of the marker genes present in the cells, identifying a gene with an unknown function based on the expression of the marker genes, and correlating an activity of one or more chemical compounds from the diverse array to the gene with unknown function to identify a function for the gene.

A yet another aspect of the invention provides a method for identifying an active compound by contacting cells with a plurality of chemical compounds, screening proteins isolated from the cells to determine expression of a set of marker proteins, and scoring the expression of the marker proteins to identify a cellular phenotype, the presence of a specific cellular phenotype being indicative of an active compound.

In yet another aspect of the invention provides a method for identifying changes in cellular proliferation by contacting cells with a plurality of chemical compounds, amplifying at least one control gene from the cells, scoring the level of expression of the control gene to determine a relative amount of cellular proliferation with respect to a level of expression of the control gene in a similar cell.

In other aspects the invention is a database representing a library of phenotypic states of cells, the database tangibly embodied on a computer-readable medium. The database includes one or more phenotype data structures, each phenotype data structure representing a phenotypic state and including at least one marker data unit representing a marker and specifying a difference in an expression level of the marker for a cell having the phenotypic state and an expression level of the marker for a biological cell not having the phenotypic state.

In another aspect the invention is a data structure representing a phenotypic state of a cell, the data structure tangibly embodied on a computer-readable medium having at least one marker data unit representing a marker and specifying a difference in an expression level of the marker for a cell having the phenotypic state and an expression level of the marker for a biological cell not having the phenotypic state, wherein the marker data unit was generated using reverse gene expression analysis.

In yet another aspect the invention is a method of determining whether a chemical compound applied to undifferentiated cells can produce a differentiated cells exhibiting a phenotype. The method involves receiving expression levels of nucleic acids of a spot of an array produced from introducing the undifferentiated cells to a chemical well containing the chemical compound; determining whether the chemical well from which the spot resulted is a dead chemical well by determining whether the resulting expression level of a housekeeping nucleic acid of the spot reaches a threshold expression level value; if the expression level of the housekeeping gene reaches the threshold value, normalizing an expression level of at least a first nucleic acid that is a marker for the phenotype; and determining whether the normalized expression level reaches a threshold level.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE DRAWINGS

FIG. 1: An example of a computer system 100 for storing and manipulating phenotype and marker information.

FIG. 2: Illustrates a process 300 that may be used by the search engine 112 to generate the search results 116.

FIG. 3: Histograms of the ratios of each phenotype (ATRA, PMA and undifferentiated) as measured by the mass spectrometer method. The histograms display the distribution of the gene intensity ratios for IL1RN relative to GAPDH (3A), NCF1 relative to GAPDH (3B), ORM1 relative to GAPDH (3C), and SPP1 relative to GAPDH (3D).

FIG. 4: Histograms of the ratios of each phenotype (ATRA, PMA and undifferentiated) as measured by the spotted microarray method. The histograms display the distribution of the gene intensity ratios for IL1RN relative to GAPDH (4A), NCF1 relative to GAPDH (4B), ORM1 relative to GAPDH (4C), and SPP1 relative to GAPDH (4D).

FIG. 5: Histograms of the ratios of each phenotype (ATRA, PMA and undifferentiated) as measured by the mass specification method. The histograms display the distribution of the gene intensity ratios for SPP1 relative to GAPDH (5A) and IL1RN relative to GAPDH (5B) using the direct gene dendrimer method.

FIG. 6: FIG. 10: Histograms of the ratios of each phenotype (ATRA, PMA and undifferentiated) as measured by the mass specification method. The histograms display the distribution of the gene intensity ratios for NCF, relative to GAPDH using the direct gene dendrimer method.

### DETAILED DESCRIPTION

The invention involves the discovery of high throughput methods of cellular analysis. Techniques such as small molecule analysis have been used as high throughput methods for screening drugs to determine the effect of a plurality of compounds on a specific biological parameter or end point. For instance small molecule libraries have been used to assess the effects of a putative ligand on a specific receptor or signal transduction process. High throughput methods for identifying gene expression information with microarrays such as Affymetrix chips have also been used. It is not feasible, however, to combine multiple known high throughput methods, i.e. to use high-density DNA microarrays as a high throughput drug screen of tens of thousands of small molecules. Such an effort would be prohibitive in both time and cost. In addition, any attempt to evaluate large chemical archives kinetically for changes in gene expression would only expand the already expensive task of high throughput screening by gene expression arrays.

Modified methods for combining multiple high throughput screening techniques have now been developed. The methods of the invention circumvent the need for identifying specific targets of a biological pathway. In general, the methods may be accomplished by defining expression signatures (referred to as sets of marker genes) for at least 2 cellular states or phenotypes, i.e. state “A” and state “B”. A library of chemical agents (or other high throughput system) may then be screened to identify compounds that induce a change in the expression signature from state “A” to state “B.” The present invention combines high throughput chemical compound library screens with gene expression signature analysis by utilizing reverse gene expression analysis and other methods described in more detail herein.

The present invention utilizes, in some aspects, a unique gene expression profile representative of a given phenotypic state that can be represented by the expression of a smaller subset of genes. The use of a smaller subset of marker genes makes feasible high throughput chemical compound library screening by utilizing gene expression signatures. The methods of the invention involve the use of a small set of marker genes selected for the ability to separate two or more phenotypic states or to specifically characterize a phenotypic state. Cells may be exposed to chemical agents in a chemical compound archive or library. A change in the expression of the marker genes serves as a proxy for a change in phenotypic state. The use of changes in cellular phenotype expands the number of targets being detected in the screen, and thereby enhances the identification of data points. It also circumvents the need for a priori knowledge of a pathway target.

Thus, in some aspects, the invention is a method of determining a gene expression profile for a cellular phenotype. The method is performed by establishing two or more sets of gene expression profiles; defining a set of marker genes that defines the differences between the two or more sets of gene expression profiles; and recording the set of marker genes in a database that defines the cellular phenotype.

Initially, the methods involve the establishment of two or more sets of gene expression profiles. These methods are described in more detail below. The gene expression profiles are utilized to develop marker gene sets which identify a phenotype. Thus the methods of the invention involve the identification of a cell signature which is useful for identifying a phenotype of a cell. A “phenotype” as used herein refers to a physiological state of a cell under a specific set of conditions.

The signature is defined by a set of marker genes. A “set of marker genes” is a minimum number of genes that is capable of identifying a phenotypic state of a cell. A set of marker genes “that is representative of a cellular phenotype” is one which includes a minimum number of genes that identify markers to demonstrate that a cell has a particular phenotype. In general, two discrete cell populations having the desired phenotypes may be examined by high density nucleic acid microarrays to produce sets of data. From these sets of genes, a smaller subset of genes called “marker genes” is used to define the difference between the two states. The minimum number of genes in a set of marker genes will depend on the particular phenotype being examined. In some embodiments the minimum number of genes is 2 or, more preferably, 5 genes. In other embodiments the minimum number of genes is 10, 15, 20, 25, 30, 35, 40, 45, 50, 55, 60, 65, 70, 75, 80, 85, 90, 95, or 1000 genes.

In addition to these marker gene sets, a control gene or set of control genes are selected that are common between the two phenotypic states in similar or equivalent degrees of gene expression. A common housekeeping gene(s) may be used as an “internal” reference or control to normalize the readout for relative differences in cell populations in the screening assay. The small molecule drug screen should not perturb the level of gene expression for the common gene(s) differently between the two phenotypic states. One example of a common gene useful in the invention is glyceraldehyde 3-phosphate dehydrogenase (GAPDH) (M33197). The expression level of the marker genes will define the phenotypic state when taken in ratio to the common gene(s). Hence, quantitation of the mRNA levels for 2 or more marker genes will be adequate to identify a new phenotypic state.

In some embodiments of the invention the method is performed with a metagene. A “metagene” is a small set of genes which are capable of faithfully representing larger sets of genes, and hence, a cellular phenotype. In one embodiment a metagene representative of a particular phenotype is the same as a set of marker genes for the particular phenotype. In other embodiments, however, the metagene is distinct from the set of marker genes for the same cellular phenotype. A metagene preferably is composed of more genes than the marker gene set. The metagene defines a detailed phenotypic state of a cell, which can distinguish such properties as the differentiation state of a cell. The metagene set includes more genes which can be used to define more aspects of the phenotype, whereas, a marker gene set includes only the most highly expressed genes, which will be characteristic of a general phenotype.

Some drug therapies work by specific action against a defined target while others act on a class of proteins. Using the methods of the invention, it is possible to use high-density DNA microarrays to map the gene expression changes for all known drugs. This comprehensive gene expression map could then be reduced down to a smaller set of metagenes. From the “all drug” metagene set one could screen an entire small molecule library for gene expression responses that fit into a specific drug category.

A metagene profile may also be used to identify the function of new genes. This may be accomplished with a collection of small molecules, which systematically induce all possible states of gene expression across the entire transcriptome. By difference analysis this set of small molecules is used to enable one to identify novel genes.

Once the marker sets or metagene sets are identified, the methods of the invention can be accomplished by high throughput screening methods. In general, the methods are performed by developing many cell samples. The cell samples may be generated by contacting a set of cells with a plurality of chemical compounds. The cells may then be analyzed to determine the effect of the chemical compounds on the phenotype of the cell. The cells may be screened to determine the level of expression of the marker genes previously identified using the methods described in more detail below. This analysis of the genes expressed in the test cells may be performed by any method which allows for high throughput screening. One method for high throughput screening is referred to herein as custom reverse microarray analysis. Another method is referred to as single base extension (SBE).

A custom reverse microarray analysis as used herein refers to a method involving spotting of the nucleic acid sample, such as target PCR amplicons, onto a slide or plate in a high density array format which enables further processing with one or more probes representative of the marker gene sets.

The custom spotted array may be performed directly on the PCR amplicons. Briefly an example of a method of custom microarray analysis is the following. The PCR fragments of each marker gene are spotted in an array format using a microarraying device. The spotted PCR fragments are then UV crosslinked and then boiled to open up the dsDNA PCR amplicons. The spotted array is then stained using a fluorescent amplifying stain such as 3DNA dendrimer staining (Genisphere) or by some other detection method such Quantum dots, Tyramide assay (NEN), resonance light scattering (RLS®, Genicon Sciences Inc.), rolling circle DNA amplification (RCA, Molecular Staging) or by NovaChip Evanesecent Resonator Slide (Novartis). The scanned image is converted into a tif file and data is extracted by any standard microarray extraction program (Arrayvision, Quantarray, Axon). Other micro-detection formats equally amenable to the spotted microarray and which could equally be adapted to reverse micro-analytical detection methods are; NanoChip® Electronic Microarray detection by Nanogen Inc. and BeadArray™ technology (fiber optic bead arrays) by Illumina, Inc.

Single base extension (SBE), which may be accomplished using a Sequenom Mass Spectrometer, involves a combination of PCR amplification and MALDI mass spectroscopy.

An exemplary method of SBE by Sequenom involves adding primers specific to the internal region of each amplified PCR fragment. The SBE reaction by Sequenom is readable in multiplex format (7 plex reaction readouts). The SBE reaction mixture is spotted in 384 well format onto a MALDI matrix coated disk and detected by mass spectrometry. The signal to noise ratio of each extended fragment after single base extension is determined relative to the good housekeeping genes.

In some embodiments, the custom reverse microarray, or other analysis, includes at least one control DNA sample. The custom reverse microarray may be screened with a plurality of different oligonucleotides that are representative of a particular set of marker genes.

The analysis using the custom reverse microarray, SBE, or other such technology involves a determination of changes in expression in genes. The genes being analyzed are either upregulated, downregulated, or remain unchanged.

“Upregulated,” as used herein, refers to increased expression of a gene. “Increased expression” refers to increasing (i.e., to a detectable extent) transcription or decreasing degradation of any of the nucleic acids of the invention, since upregulation of any of these processes results in concentration/amount increase of the transcript (mRNA) encoded by the gene. Conversely, downregulation or decreased expression refers to decreased expression of a gene. The upregulation or downregulation of gene expression can be directly determined by detecting an increase or decrease, respectively, in the level of mRNA for the gene, using any suitable means known to the art, and optionally using hybridization and nucleic acid array technology, and in comparison to controls.

As used herein, a subject is a human or a non-human mammal, e.g, a dog, cat, horse, cow, pig, sheep, goat, monkey, rabbit, rat, mouse, etc. In many embodiments human nucleic acids, polypeptides, and human subjects are used.

It is also possible that the gene expression may provide a “guide” for the identification of a specific set of protein targets whose concentration and physical state are also sufficient to separate the two phenotypes. Thus, in some embodiments the custom reverse microarray may also be a peptide based array. The peptide arrays provided for use herein may comprise either the peptides or polypeptides isolated from the test cells being examined. These arrays may be screened using binding partners of the peptides encoded by the set of marker genes identified using the methods described below The binding partners could commonly comprise antibodies or antibody fragments that bind specifically to peptides or polypeptides encoded by the marker genes. The peptide based custom reverse microarray analysis may be used alongside of or in some circumstances in place of the nucleic acid based custom reverse microarray. One advantage of using both a peptide based and a nucleic acid based custom reverse microarray is that a combination of protein and mRNA expression may provide a more detailed map of the phenotypic characteristics or state of a cell than either form of analysis alone.

The probes that are used to identify the marker genes of the custom reverse array are unique fragments. A “unique fragment,” as used herein with respect to a nucleic acid is one that is a ‘signature’ for the larger nucleic acid. For example, the unique fragment is long enough to assure that its precise sequence is not found in molecules within the human genome outside of the sequence for each nucleic acid listed herein. Those of ordinary skill in the art may apply no more than routine procedures to determine if a fragment is unique within the human genome.

As will be recognized by those skilled in the art, the size of the unique fragment will depend upon its conservancy in the genetic code. Thus, some regions will require longer segments to be unique while others will require only short segments, typically between 12 and 32 nucleotides long (e.g. 12, 13, 14, 15, 16, 17, 18, 19, 20, 21, 22, 23, 24, 25, 26, 27, 28, 29, 30, 31 and 32 bases) or more, up to the entire length of each of the disclosed sequences. Those skilled in the art are well versed in methods for selecting such sequences, typically on the basis of the ability of the unique fragment to selectively distinguish the sequence of interest from other sequences in the human genome of the fragment to those on known databases typically is all that is necessary, although in vitro confirmatory hybridization and sequencing analysis may be performed.

Additionally, unique fragments of both nucleic acids and polypeptides or peptides encoded by those nucleic acids are useful in the microarrays described in more detail below. It is preferred that the nucleic acids and peptides used to identify markers be unique to that marker in order to reduce non-specific binding.

Some examples of discrete phenotypic differences that can be evaluated using the methods of the invention are: (1) cancer vs. non-cancer (2) metastatic vs. non-metastatic cancer (3) cancers that are resistant to radiation vs. cancers that are susceptible to radiation (4) cancers that are susceptible to chemotherapy vs. chemotherapy resistant cancers (5) cancers that release angiogenic factors vs. cancers that do not release angiogenic factors (6) cell populations which have a positive drug response vs. cells that have a negative drug response (7) “enhancer assays” wherein one cell has a response to a given chemical agent but that the response is enhanced by addition of a secondary agent. Many others will also be useful.

Thus, the screening methods may be used for identifying therapeutic agents or validating the efficacy of agents. Agents of either known or unknown identity can be analyzed for their effects on gene expression in cells using methods such as those described herein. Briefly, purified populations of cells are exposed to the plurality of chemical compounds, preferably in an in vitro culture high throughput setting, and optionally after set periods of time, the entire cell population or a fraction thereof is removed and mRNA is harvested therefrom. Either mRNA or cDNA is then analyzed for expression of marker genes using methods such as those described herein. Hybridization or other expression level readouts may be then compared to the marker gene data. These methods can be used for identifying novel agents, as well as confirming the identity of agents that are suspected of playing a role in regulation of cellular phenotype.

The methods of the invention allows for subjects to be screened and potentially characterized according to their ability to respond to a plurality of drugs. For instance, cells of a subject, e.g., cancer cells, may be removed and exposed to a plurality of putative therapeutic compounds, e.g., anti-cancer drugs, in a high throughput manner. The nucleic acids of the cells may then be screened using the methods described herein to determine whether marker genes indicative of a particular phenotype are expressed in the cells. These techniques can be used to optimize therapies for a particular subject. For instance, a particular anti-cancer therapy may be more effective against a particular cancer cell from a subject. This could be determined by analyzing the genes expressed in response to the plurality of compounds. Likewise a therapeutic agent with minimal side effects may be identified by comparing the genes expressed in the different cells with a marker gene set that is indicative of a phenotype not associated with a particular side effect. Additionally, this type of analysis can be used to identify subjects for less aggressive, more aggressive, and generally more tailored therapy to treat a disorder.

The methods are also useful for determining the effect of multiple drugs or groups of drugs on a cellular phenotype. For instance it is possible to perform combined chemical genomic screens to identify a synergistic or other combined effect arising from combinations of drugs. One set of drugs that induces a first set of marker genes indicative of a phenotype, while another drug induces an second set of marker genes. When the two sets of drugs are combined they may act to achieve a collective phenotypic change, exemplified by a third set of marker genes. Additionally the methods could be used to assess complex multidrug effects on cell types. For instance, some drugs when used in combination produce a combined toxic effect. It is possible to perform the screen to identify marker genes associated with the toxic phenotype. Existing compounds could be screened for there ability to “trip” the signal signature of toxic effect, by monitoring the marker genes associated with the toxic phenotype.

The methods may also be used to enhance therapeutic strategies. For instance, oncolytic therapy involves the use of viruses to selectively lyse cancer cells. A set of marker genes which identify a gene expression signature favorable to selective viral infection can be identified. Using this set of marker genes, drugs can be found which favor or enable selective viral infectivity in order to enhance the therapeutic benefit.

Thus, the methods of the invention are useful for screening multiple compounds. For instance, the methods are useful for screening libraries of molecules, FDA approved drugs, and any other sets of compounds. Preferably the methods are used to screen at least 20 or 30 compounds, and more preferably, at least 50 compounds. In some embodiments, the methods are used to screen more than 96, 384, or 1536 compounds at a time.

In one embodiment, the methods of the invention are useful for screening FDA approved drugs. An FDA approved drug is any drug which has been approved for use in humans by the FDA for any purpose. This is a particularly useful class of compounds to screen because it represents a set of compounds which are believed to be safe and therapeutic for at least one purpose. Thus, there is a high likelihood that these drugs will at least be safe and possibly be useful for other purposes. FDA approved drugs are also readily commercially available from a variety of sources.

A “library of molecules” as used herein is a series of molecules displayed such that the compounds can be identified in a screening assay. The library may be composed of molecules having common structural features which differ in the number or type of group attached to the main structure or may be completely random. Libraries are meant to include but are not limited to, for example, phage display libraries, peptides-on-plasmids libraries, polysome libraries, aptamer libraries, synthetic peptide libraries, synthetic small molecule libraries and chemical libraries. Methods for preparing libraries of molecules are well known in the art and many libraries are commercially available. Libraries of interest include synthetic organic combinatorial libraries. Libraries, such as, synthetic small molecule libraries and chemical libraries. The libraries can also comprise cyclic carbon or heterocyclic structure and/or aromatic or polyaromatic structures substituted with one or more functional groups. Libraries of interest also include peptide libraries, randomized oligonucleotide libraries, and the like. Degenerate peptide libraries can be readily prepared in solution, in immobilized form as bacterial flagella peptide display libraries or as phage display libraries. Peptide ligands can be selected from combinatorial libraries of peptides containing at least one amino acid. Libraries can be synthesized of peptoids and non-peptide synthetic moieties. Such libraries can further be synthesized which contain non-peptide synthetic moieties which are less subject to enzymatic degradation compared to their naturally-occurring counterparts.

Small molecule combinatorial libraries may also be generated. A combinatorial library of small organic compounds is a collection of closely related analogs that differ from each other in one or more points of diversity and are synthesized by organic techniques using multi-step processes. Combinatorial libraries include a vast number of small organic compounds. One type of combinatorial library is prepared by means of parallel synthesis methods to produce a compound array. A “compound array” as used herein is a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address. Examples of parallel synthesis mixtures and parallel synthesis methods are provided in U.S. Pat. No. 5,712,171 issued Jan. 27, 1998.

One type of library, which is known as a phage display library, includes filamentous bacteriophage which present a library of peptides or proteins on their surface. Phage display libraries can be particularly effective in identifying compounds which induce a desired effect in cells. Briefly, one prepares a phage library (using e.g. ml3, fd, lambda or T7 phage), displaying inserts from 4 to about 80 amino acid residues using conventional procedures. The inserts may represent, for example, a completely degenerate or biased array. DNA sequence analysis can be conducted to identify the sequences of the expressed polypeptides. The minimal linear peptide or amino acid sequence that have the desired effect on the cells can be determined. One can repeat the procedure using a biased library containing inserts containing part or all of the minimal linear portion plus one or more additional degenerate residues upstream or downstream thereof.

For certain embodiments of this invention, e.g., where phage display libraries are employed, a preferred vector is filamentous phage, though other vectors can be used. Vectors are meant to include, e.g., phage, viruses, plasmids, cosmids, or any other suitable vector known to those skilled in the art. The vector has a gene, native or foreign, the product of which is able to tolerate insertion of a foreign peptide. By gene is meant an intact gene or fragment thereof. Filamentous phage are single-stranded DNA phage having coat proteins. Preferably, the gene that the foreign nucleic acid molecule is inserted into is a coat protein gene of the filamentous phage. Examples of coat proteins are gene III or gene VIII coat proteins. Insertion of a foreign nucleic acid molecule or DNA into a coat protein gene results in the display of a foreign peptide on the surface of the phage. Examples of filamentous phage vectors which can be used in the libraries are fUSE vectors, e.g., fUSE1 fUSE2, fUSE3 and fUSE5, in which the insertion is just downstream of the pill signal peptide. Smith and Scott, Methods in Enzymology 217:228-257 (1993).

By recombinant vector it is meant a vector having a nucleic acid sequence which is not normally present in the vector. The foreign nucleic acid molecule or DNA is inserted into a gene present on the vector. Insertion of a foreign nucleic acid into a phage gene is meant to include insertion within the gene or immediately 5′ or 3′ to, respectively, the beginning or end of the gene, such that when expressed, a fusion gene product is made. The foreign nucleic acid molecule that is inserted includes, e.g., a synthetic nucleic acid molecule or a fragment of another nucleic acid molecule. The nucleic acid molecule encodes a displayed peptide sequence. A displayed peptide sequence is a peptide sequence that is on the surface of, e.g. a phage or virus, a cell, a spore, or an expressed gene product.

In certain embodiments, the libraries may have at least one constraint imposed upon their members. A constraint includes, e.g., a positive or negative charge, hydrophobicity, hydrophilicity, a cleavable bond and the necessary residues surrounding that bond, and combinations thereof. In certain embodiments, more than one constraint is present in each of the broader sequences of the library.

In addition to the basic libraries, the methods can also be used to screen combinations of drugs. Thus, more than one type of drug can be contacted with each cell.

In other aspects of the invention, the cells do not necessarily need to be contacted with any compounds. The cells may be analyzed for phenotypic status based on environmental condition, such as in vivo or in vitro conditions. It is possible to analyze the differentiation state or tumorigenic state of a cell using the marker gene sets or metagenes of the invention. Thus, a cell may be subjected to conditions in vitro or in vivo and then analyzed for differentiation status.

Additionally, it is possible to screen sets of compounds to identify particular dosages effective at producing a phenotypic state in a cell. For instance, one or more drugs could be contacted with the cells at a variety of dosages over a large range. When the level of marker genes expressed in each of the cells is assessed, it will be possible to identify an optimum dosage for producing a particular phenotypic state of the cell. Additionally, if some markers are associated with the production of undesirable side effects, such as production of cytotoxic factors, then an optimum drug, combination of drug or dosage of drug can be identified using the methods of the invention.

The methods of the invention are useful for assaying the effect of compounds on cells or for analyzing the phenotypic status of a cell. The methods may be used on any type of cell known in the art. For instance the cell may be a cultured cell line or a cell isolated from a subject (i.e. in vivo cell population). The cell may have any phenotypic property, status or trait. For instance, the cell may be a normal cell, a cancer cell, a genetically altered cell, etc.

Cancers include, but are not limited to, basal cell carcinoma, biliary tract cancer; bladder cancer; bone cancer; brain and CNS cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia; liver cancer; lung cancer (e.g., small cell and non-small cell); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas. Some cancer cells are metastatic cancer cells.

“Normal cells” as used herein refers any cell, including but not limited to mammalian, bacterial, plant cells, that is a non-cancer cell, non-diseased, or a non-genetically engineered cell. Mammalian cells include but are not limited to mesenchymal, parenchymal, neuronal, endothelial, and epithelial cells.

A “genetically altered cell” as used herein refers to a cell which has been transformed with an exogenous nucleic acid.

As mentioned above, the marker gene sets may be developed from gene expression profiles. The gene expression profiles may be created using a variety of high throughput technologies such as high-density DNA microarrays, real-time PCR or SAGE (Serial Analysis of Gene Expression). Analysis by the SAGE method is conducted with short sequence tags that can identify unique transcripts. The number of these tags are directly related to the expression level of the unique transcript. These tags can be linked together to be cloned and sequenced. This as well as other methods known to those skilled in the art may be utilized to perform initial gene expression profiling.

In addition to gene expression profiles, protein expression profiles may be used. The relevant profile for proteomics can be determined by a number of methods such as differences in protein concentration or by post-translational modifications such as methylation, phosphorylation and glycosylation. For purposes of brevity the term “gene expression profile” is used throughout the description. But this aspect can also be applied to protein expression profiles. DNA and protein microarray technology has been described in the art.

In general solid-phase arrays are composed of a plurality of distinct nucleic acid molecules, expression products thereof, or fragments thereof fixed to a solid substrate. Standard hybridization techniques of microarray technology are utilized to assess patterns of nucleic acid expression. Microarray technology, which is also known by other names including DNA chip technology, gene chip technology, and solid-phase nucleic acid array technology, is well known to those of ordinary skill in the art and is based on, but not limited to, obtaining an array of identified nucleic acid probes on a fixed substrate, labeling target molecules with reporter molecules (e.g., radioactive, chemiluminescent, or fluorescent tags such as fluorescein, Cye3-dUTP, or Cye5-dUTP), hybridizing target nucleic acids to the probes, and evaluating target-probe hybridization. A probe with a nucleic acid sequence that perfectly matches the target sequence will, in general, result in detection of a stronger reporter-molecule signal than will probes with less perfect matches. Many components and techniques utilized in nucleic acid microarray technology are presented in The Chipping Forecast, Nature Genetics, Vol. 21, January 1999, the entire contents of which is incorporated by reference herein.

Microarray substrates may include but are not limited to glass, silica, aluminosilicates, borosilicates, metal oxides such as alumina and nickel oxide, various clays, nitrocellulose, or nylon. In some embodiments, the nucleic acid molecules are fixed to the solid substrate by covalent bonding. Probes generally are selected from the group of nucleic acids including, but not limited to: DNA, genomic DNA, cDNA, and oligonucleotides; and may be natural or synthetic. Oligonucleotide probes preferably are 20 to 25-mer oligonucleotides and DNA/cDNA probes preferably are 500 to 5000 bases in length, although other lengths may be used. Appropriate probe length may be determined by one of ordinary skill in the art by following art-known procedures. Probes may be purified to remove contaminants using standard methods known to those of ordinary skill in the art such as gel filtration or precipitation. Preferably the nucleic acids fixed to the solid support are or comprise unique fragments.

Optionally, the microarray substrate may be coated with a compound to enhance synthesis of the probe on the substrate. Such compounds include, but are not limited to, oligoethylene glycols. In another embodiment, coupling agents or groups on the substrate may be used to covalently link the first nucleotide or oligonucleotide to the substrate. These agents or groups may include, but are not limited to: amino, hydroxy, bromo, and carboxy groups. These reactive groups are preferably attached to the substrate through a hydrocarbyl radical such as an alkylene or phenylene divalent radical, one valence position occupied by the chain bonding and the remaining attached to the reactive groups. These hydrocarbyl groups may contain up to about ten carbon atoms, preferably up to about six carbon atoms. Alkylene radicals are usually preferred containing two to four carbon atoms in the principal chain. These and additional details of the process are disclosed, for example, in U.S. Pat. No. 4,458,066, which is incorporated by reference.

The probes may be synthesized directly on the substrate in a predetermined grid pattern using methods such as light-directed chemical synthesis, photochemical deprotection, or delivery of nucleotide precursors to the substrate and subsequent probe production.

Additionally, the substrate may be coated with a compound to enhance binding of the probe to the substrate. Such compounds include, but are not limited to: polylysine, amino silanes, amino-reactive silanes, or chromium. In this embodiment, presynthesized probes are applied to the substrate in a precise, predetermined volume and grid pattern, utilizing a computer-controlled robot to apply probe to the substrate in a contact-printing manner or in a non-contact manner such as ink jet or piezo-electric delivery. Probes may be covalently linked to the substrate with methods that include, but are not limited to, UV-irradiation or covalent coupling by chemically activated slides. In another embodiment probes are linked to the substrate with heat.

Nucleic acids that can be applied to the array may be natural or synthetic. In certain embodiments of the invention, one or more control nucleic acid molecules are attached to the substrate. Preferably, control nucleic acid molecules allow determination of factors including but not limited to: nucleic acid quality and binding characteristics; reagent quality and effectiveness; hybridization success; and analysis thresholds and success. Control nucleic acids may include, but are not limited to, expression products of genes such as housekeeping genes or fragments thereof.

To select a set of markers useful according to the invention, the expression data generated by, for example, microarray analysis of gene expression, is preferably analyzed to determine which genes are significantly differentially expressed in response to a set of putative active compounds. The significance of gene expression can be determined using any standard statistical computer software that can discriminate significant differences in expression, such as ScanAnalyze, Cluster and TreeView (M. Eisen), Cluster (G. Sherlock) or Permax computer software. Permax performs permutation 2-sample t-tests on large arrays of data. For high dimensional vectors of observations, the Permax software computes t-statistics for each attribute, and assesses significance using the permutation distribution of the maximum and minimum overall attributes. The main uses include determining the attributes (genes) that are the most different between stimulated and unstimulated samples, or in other embodiments between different subsets of cells, or in yet other embodiments, between different patients, measuring “most different” using the value of the t-statistics, and their significance levels. Optimized methods for detecting differences in gene expression and data analysis are described in more detail below.

Although it is preferred that the expression profile of markers is developed using nucleic acid based microarrays, an expression profile of markers (i.e. set of marker proteins) may also be determined using protein measurement methods. The relevant profile for proteomics can be differentiated by differences in protein concentration or post-translational modifications such as methylation, phosphorylation or glycosylation. Methods of specifically and quantitatively measuring proteins include, but are not limited to mass spectroscopy-based methods such as peptide microarrays, surface enhanced laser desorption ionization (SELDI; e.g., Ciphergen ProteinChip System), non-mass spectroscopy-based methods, and immunohistochemistry-based methods such as 2-dimensional gel electrophoresis.

SELDI methodology may, through procedures known to those of ordinary skill in the art, be used to vaporize microscopic amounts of protein and to create a “fingerprint” of individual proteins, thereby allowing simultaneous measurement of the abundance of many proteins in a single sample. Preferably, SELDI-based assays may be utilized to characterize cellular responses as well as stages of particular conditions, or particular therapy regimens. Such assays preferably include, but are not limited to the following examples. Gene products discovered by RNA microarrays may be selectively measured by specific (antibody, hapten or aptamer mediated) capture to the SELDI protein disc (e.g., selective SELDI).

As stated previously, the current invention involves a method for effectively combining the power of gene expression DNA arrays with high throughput drug screens. Experimentally, an example of the method can be described in four steps: (1) identification of a set of marker genes representative of a desirable phenotypic state (2) optionally contacting the cells with putative therapeutic agents followed by amplification of the marker genes from the cells by PCR in a high throughput format (3) quantifying the PCR reactions by a high throughput detection method such as mass spectroscopy or by custom microarray (4) scoring the defined changes in the levels.

Several statistical methods can be used to identify marker genes from the gene expression profile that are characteristic of the differences between the two phenotypic states, e.g., gene cluster (self organized maps), nearest neighbor analysis, or by hierarchical tree clustering. In some embodiments, the gene sets are essentially “binary” in that the difference between the mRNA levels is either “all on” or “all off”. In other embodiments the data consists of non-binary mRNA levels where the mRNA is expressed in both states but differs between states by an amount quantifiable. Methods for analyzing the data from binary and non-binary systems is described herein.

An analysis scheme, containing several algorithms to combine and analyze the data from replicate screens of the plurality of samples has been developed. This analysis increases the accuracy of the methods to indicate the phenotype of the cells being screened. The method may be accomplished using control samples. For instance, the methods may involve the analysis of cells which are treated with known chemical compounds causing a known change in phenotype. The measured expression levels of several genes from the control samples may be assessed. Since these cells have a known phenotypic change, it is possible to predict the effect on the marker genes. For instance the control cells may be treated with chemical compounds that are known to cause a desired type of differentiation. Then genes that are predictive of that state of differentiation may be utilized as markers. It has been discovered that the use of ratios of measured expression levels in this analysis will help improve the accuracy of the data output. In general the ratio is arranged with a numerator including a value for one set of expression levels and a denominator with a value of another set of expression levels. The values represent the expression levels of genes of control cells, optionally spotted on different plates or analyzed with different probes. The value for the numerator represents the genes which are differentially expressed in the two cells states but have higher expression in one of the two cell states (i.e. the differentiated or tumorigenic state). The value for the denominator represents either the genes which are differentially expressed in the two cells states but have higher expression in the second of the two cell states (i.e. the undifferentiated or non-tumorigenic state) or else a housekeeping gene that is uniformly expressed in both states of the cell. These ratios form normalized expression values that are consistent from plate to plate and well to well.

Another step in the data analysis program involves filtering to eliminate dead wells from subsequent analysis steps. This step is useful for removing background noise from the analysis. When using ratios a small expression value (nominally zero with some measurement noise) may produce a large ratio which would be improperly indicative of a particular cell state, i.e. a differentiated cell. In order to avoid this we have developed two approaches to filtering.

The first approach works with the SBE/MALDI-TOF readout method and uses a score generated by the SBE/MALDI-TOF machine that gives the likelihood that the measured peak has the characteristics of a real peak. This score has values between 0 and 1. We typically filter out wells with scores for the housekeeping gene below 0.5.

The second approach calculates statistics for the expression levels of the housekeeping genes in the negative control samples and filters out samples with housekeeping gene expression levels below the mean plus standard deviation of the negative control samples. With microarray readout data, there is one additional step. Since a preferred method involves the samples being spotted in duplicate on the slides, the duplicate measurements are combined into a single expression value so the two readout values can use identical processing steps. One method involves utilizing the average of the duplicate spots as long as they both pass the filtering test. If one of the spots did not pass the filtering test, then it is eliminated from the analysis. If both spots did not pass the filtering, then the sample is considered to be filtered out (i.e., containing dead cells).

The normalized expression values (expression ratios describe above) are then converted into a measure of the likelihood of the well containing differentiated cells. At least two methods may be utilized to perform this stage of analysis. The first method uses the measured expression levels from the control samples to perform a threshold analysis. The threshold for identifying a sample as having a specific phenotype is generated from the control samples spotted on the same plate and from control samples spotted on other plates. The threshold is optimized to minimize the overall error cost, Cₑ, where different costs can be assigned to the error of identifying a phenotype (i.e., calling a differentiated sample undifferentiated and vice versa) as follows:

Cₑ=C_{d}*E_{d}+Cᵤ*Eᵤ

where

C_{d}—the cost associated with calling a well with differentiated cells undifferentiated.

Cᵤ—the cost associated with calling a well with undifferentiated cells differentiated.

E_{d}—the number of differentiated control wells miscalled as undifferentiated.

Eᵤ—the number of undifferentiated control wells miscalled as differentiated.

The value of the costs are determined by making a tradeoff between the number of false positives and false negatives. Generally, it is desirable to place a higher cost on identifying a false negative (i.e. calling a differentiated sample an undifferentiated sample) because it is of interest to not miss identifying new differentiating compounds. Also it is relatively easy to perform additional screen to eliminate any false positives. Additionally, the errors can be weighted to bias the threshold setting to using the controls on the target plate as opposed to a separate plate of controls. This is desirable because the controls on the target plate would have experienced the same processing conditions whereas other plates may have experience slightly different processing conditions.

A second approach to performing this analysis uses a probability based MAP (maximum a posteriori) criterion:

$$\frac{P ( {H}_{1} ) {f}_{Y \vert {H}_{1}} ( y \vert {H}_{1} )}{P ( {H}_{0} ) {f}_{Y \vert {H}_{0}} ( y \vert {H}_{0} )} \begin{matrix}\begin{matrix}\begin{matrix}{H}_{1} \\ >\end{matrix} \\ <\end{matrix} \\ {H}_{0}\end{matrix} 1$$

where f(y|H) is the probability density for the expression level given the hypothesis H, P(H) is the a priori probability of hypothesis H, and H₀ and H₁ represent the two hypotheses of different phenotypic states (i.e., undifferentiated and differentiated). In this case, a multidimensional Gaussian model may be used for the probability densities where the parameters for the models would come from training the sufficient statistics using the log-expression ratios for the control samples. The log-expression ratios are used because they fit a symmetric Gaussian distribution better. This method of using the MAP criterion has the advantage that it can work with multi-class problems such as the three class undifferentiated, neutrophil differentiated, and monocyte differentiated, described in the Examples. In this case, we would just assign a log likelihood score to each of the classes where the log likelihood is defined by:

log(P(Hᵢ)ƒ_{Y|Hi}(y|Hᵢ))

and then pick the class with the highest log likelihood. The log likelihood would also provide a measure of the confidence of the classification.

In some embodiments it is desirable to perform multiple replicates of an experiment to reduce false positives and to combine the results from multiple plates. One method involves combining replicate data after performing either the threshold analysis or classification with the MAP criterion. A “hit” obtained using these methods is only considered to be real if it occurs on all of the replicate plates.

The information generated according to the methods described above, in particular the information about expression levels of markers (e.g., nucleic acid sequences or peptides), can be included in a data structure (e.g., as part of a database), on a computer-readable medium, where the information may be correlated with other information pertaining to the markers, for example, information about phenotypic states.

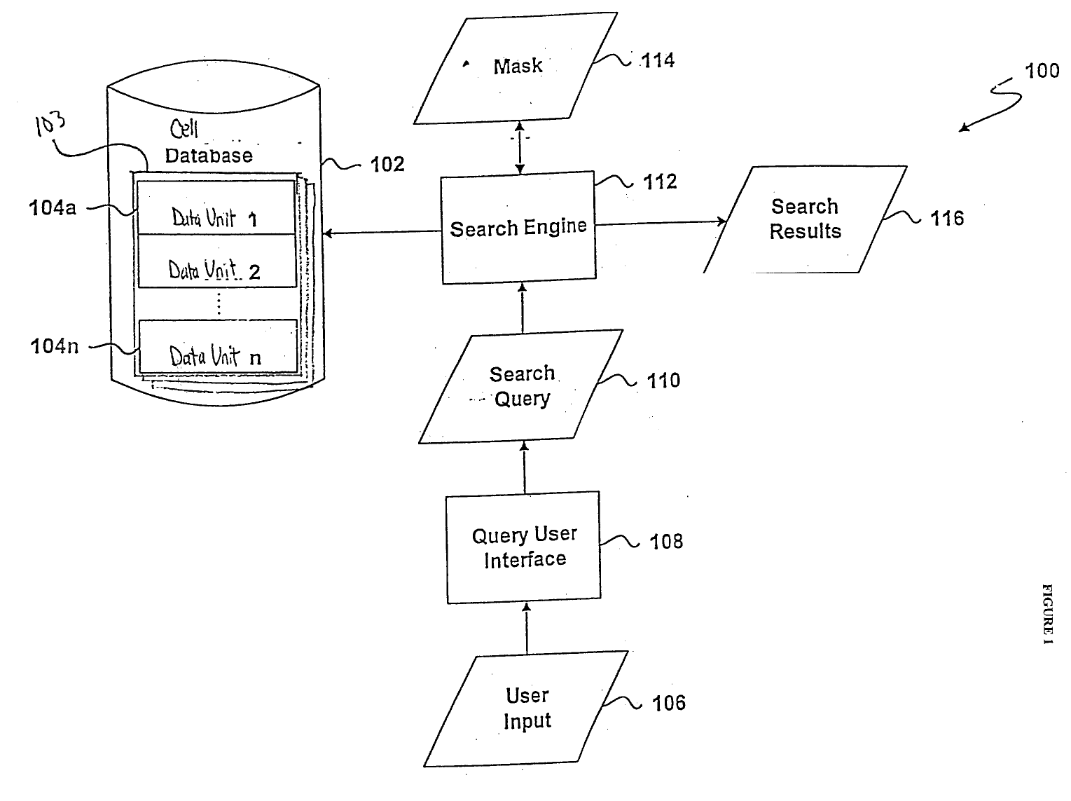

FIG. 1 shows an example of a computer system 100 for storing and manipulating phenotype and marker information. The computer system 100 includes a cell phenotype database 102 which includes a plurality cell phenotype data structures 103. Each cell phenotype data structure includes a plurality of marker data units (e.g., records or objects) 104a-n, each marker data unit storing information corresponding to a marker. Each of the marker data units 104a-n may store information about expression levels of a particular marker associated with the phenotype represented by the phenotype data structure 103, as well as other related information.

The information stored in a phenotype data structure 103 may be generated in any of a variety of ways. For example, such information may be generated using high-density DNA microarrays, as described above, or may be generated from the results of the four steps of the method described above and the subsequent data analysis.

If the cell phenotype data structure 103 is a table of a relational database and each of the marker data units is represented as a row of the table, for each row, one of the information fields included in the row or a combination of two or more of the information fields may serve as a key that uniquely identifies the row. For example, a row may include a marker identifier field that serves as a key for the row.

Cell phenotype data structure 103 may be implemented in any of a variety of ways, for example, as part of a database. For example, cell data structure 103 may be implemented as part of: a file system including one or more flat-file data structures, where data is organized into data units separated by delimiters; a relational database where data is organized into data units stored in tables; an object-oriented database where data is organized into data units stored as objects; another type of database; or any combination of these types of databases.

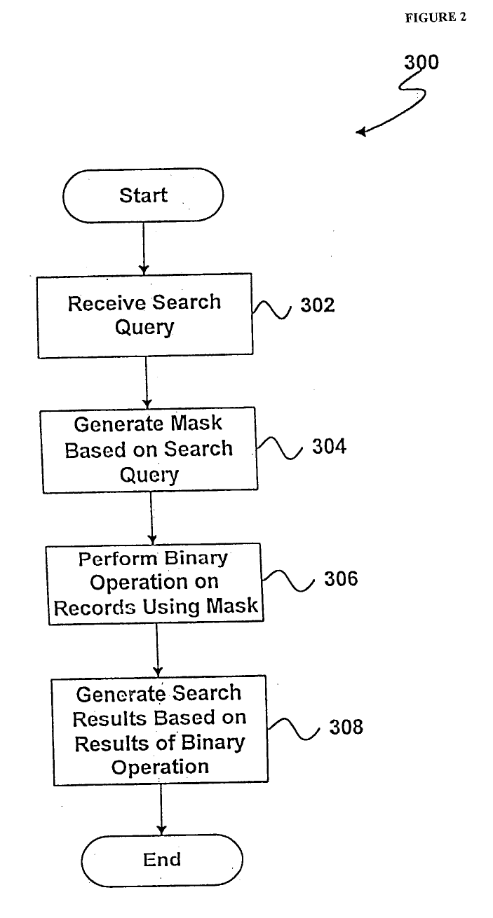

The cell phenotype data structure of 103 may be distributed across multiple data structures, where one or more of these data structures are linked. Further, any information field of a marker data unit 104 may be used as an entry in an index data structure that indexes markers sharing common attributes. Such an index structure may have a structure similar to cell data structure 103 and can be searched as part of a query, for example, as described below in more detail in relation to FIGS. 1 and 2.

The amount of information stored for each data unit 103, the number of data units 103, and the number of fields of a data unit 103 that are indexed may vary. Further, an information field may include one or more fields itself, and each of these fields themselves may include more fields, etc. Information fields may store any kind of value that is capable of being stored in a computer readable medium such as, for example, a string of characters, a binary value, a hexadecimal value, an integral decimal value, or a floating point value.

A user may perform a query on the cell phenotype database 102 for any of a variety of purposes, for example, as described in the methods set forth above: to identify a cell phenotype; to identify a subject; to evaluate a subject; to identify an agent; or for any of a variety of other purposes. To execute a query, one or more user-input expression levels of a marker or other phenotype information may be compared against marker data units (e.g., data units 104) of one or more phenotype data structures (e.g., data structures 103) to determine which data structures satisfy (i.e., match) the user-input levels of expression (i.e., the search criteria). Further analysis may be performed to determine which data structure best matches the search criteria.

Referring to FIG. 1, a user may provide, to a query user interface 108, user input 106 indicating marker or phenotype information for which to search. The user input 106 may indicate one or more expression levels of a marker or other phenotype information for which to search, using a standard character-based notation. The query user interface 108 may provide a graphical user interface (GUI) which allows the user to select from a list of types of accessible marker or phenotype information using an input device such as a keyboard or a mouse.

The query user interface 108 generates a search query 110 based on the user input 106. A search engine 112 receives the search query 110 and generates a mask 114 based on the search query. Example formats of the mask 114 and ways in which the mask 114 may be used to determine whether the marker information specified by the mask 114 matches marker information of cell data structures 103 in the cell database 102 are described in more detail below.

The search engine 112 determines whether the information specified by the mask 114 matches phenotype information stored in the cell phenotype database 102. As a result of the search, the search engine 112 generates search results 116 indicating whether the cell phenotype database 102 includes one or more cell phenotype data structures 103 having the phenotype information specified by the mask 114. More specifically, the search engine 112 may generate search results 116 indicating whether one or more cell phenotype data structures 103 have data units 104 that include marker information matching the marker information specified by the mask 114. The search results 116 also may indicate which cell phenotype data structures in the cell database 102 have the phenotype information specified by the mask 114.

For example, if the user input 106 specifies expression levels for each of the following markers: TNF-beta, CXCL-11, CCL-05, CCL-04, CXCL-10, BFL-1, CFLA and IL-1-beta, the search results 116 may indicate which cell phenotype data structure 103 in the cell phenotype database 102 include marker data units 104 that include marker information matching the expression levels of the markers specified by the user input 106. The search engine 112 or another element of the system 100 may be configured with the definition of a match. For example, a match may be defined as an expression level stored in a marker data unit 104 for a marker that has a value within ±5% of the expression level defined for the marker in the user input 106.

FIG. 2 illustrates a process 300 that may be used by the search engine 112 to generate the search results 116. The search engine 112 receives the search query 110 from the query user interface 108 (step 302). The search engine 112 generates the mask 114 generated based on the search query 110 (step 304). The search engine 112 performs a binary operation on one or more of the data units 104a-n in the cell phenotype database 102 using the mask 114 (step 306). The search engine 112 generates the search results 116 based on the results of the binary operation performed in step 306 (step 308).

The methods, steps, systems, and system elements described above may be implemented using a computer system, such as the various embodiments of computer systems described below. The methods, steps, systems, and system elements described above are not limited in their implementation to any specific computer system described herein, as many other different machines may be used.

Such a computer system may include several known components and circuitry, including a processing unit (i.e., processor), a memory system, input and output devices and interfaces, transport circuitry (e.g., one or more busses), a video and audio data input/output (I/O) subsystem, special-purpose hardware, as well as other components and circuitry, as described below in more detail. Further, the computer system may be a multi-processor computer system or may include multiple computers connected over a computer network.

The computer system may include a processor, for example, a commercially available processor such as one of the series x86, Celeron and Pentium processors, available from Intel, similar devices from AMD and Cyrix, the 680×0 series microprocessors available from Motorola, and the PowerPC microprocessor from IBM. Many other processors are available, and the computer system is not limited to a particular processor.

A processor typically executes a program called an operating system, of which WindowsNT, Windows95 or 98, UNIX, Linux, DOS, VMS, MacOS and OS8 are examples, which controls the execution of other computer programs and provides scheduling, debugging, input/output control, accounting, compilation, storage assignment, data management and memory management, communication control and related services. The processor and operating system together define a computer platform for which application programs in high-level programming languages are written. The computer system is not limited to a particular computer platform.

The computer system may include a memory system, which typically includes a computer readable and writeable non-volatile recording medium, of which a magnetic disk, optical disk, a flash memory and tape are examples. Such a recording medium may be removable, for example, a floppy disk, read/write CD or memory stick, or may be permanent, for example, a hard drive. Such a recording medium stores signals, typically in binary form (i.e., a form interpreted as a sequence of one and zeros). A disk (e.g., magnetic or optical) has a number of tracks on which such signals may be stored. Such signals may define a program, e.g., an application program, to be executed by the microprocessor, or information to be processed by the application program.

The memory system of the computer system also may include an integrated circuit memory element, which typically is a volatile, random access memory such as a dynamic random access memory (DRAM) or static memory (SRAM). Typically, in operation, the processor causes programs and data to be read from the non-volatile recording medium into the integrated circuit memory element, which typically allows for faster access to the program instructions and data by the processor than does the non-volatile recording medium.

The processor generally manipulates the data within the integrated circuit memory element in accordance with the program instructions and then copies the manipulated data to the non-volatile recording medium after processing is completed. A variety of mechanisms are known for managing data movement between the non-volatile recording medium and the integrated circuit memory element, and the computer system that implements the methods; steps, systems and system elements described above in relation to FIGS. 1 and 2 is not limited thereto. The computer system is not limited to a particular memory system.

At least part of such a memory system described above may be used to store one or more of the data structures described above in relation to FIGS. 1 and 2. For example, at least part of the non-volatile recording medium may store at least part of a database that includes one or more of such data structures. Such a database may be any of a variety of types of databases, for example, a file system including one or more flat-file data structures where data is organized into data units separated by delimiters, a relational database where data is organized into data units stored in tables, an object-oriented database where data is organized into data units stored as objects, another type of database, or any combination thereof.

The computer system may include a video and audio data I/O subsystem. An audio portion of the subsystem may include an analog-to-digital (A/D) converter, which receives analog audio information and converts it to digital information. The digital information may be compressed using known compression systems for storage on the hard disk to use at another time. A typical video portion of the I/O subsystem may include a video image compressor/decompressor of which many are known in the art. Such compressor/decompressors convert analog video information into compressed digital information, and vice-versa. The compressed digital information may be stored on hard disk for use at a later time.

The computer system may include one or more output devices. Example output devices include a cathode ray tube (CRT) display, liquid crystal displays (LCD) and other video output devices, printers, communication devices such as a modem or network interface, storage devices such as disk or tape, and audio output devices such as a speaker.

The computer system also may include one or more input devices. Example input devices include a keyboard, keypad, track ball, mouse, pen and tablet, communication devices such as described above, and data input devices such as audio and video capture devices and sensors. The computer system is not limited to the particular input or output devices described herein.

The computer system may include specially programmed, special purpose hardware, for example, an application-specific integrated circuit (ASIC). Such special-purpose hardware may be configured to implement one or more of the methods, steps and systems described above.

The computer system and components thereof may be programmable using any of a variety of one or more suitable computer programming languages. Such languages may include procedural programming languages, for example, C, Pascal, Fortran and BASIC, object-oriented languages, for example, C++, Java and Eiffel and other languages, such as a scripting language or even assembly language.

The methods, steps and systems described above may be implemented using any of a variety of suitable programming languages, including procedural programming languages, object-oriented programming languages, other languages and combinations thereof, which may be executed by such a computer system. Such methods and steps may be implemented as separate modules of a computer program, or may be implemented individually as separate computer programs. Such modules and programs may be executed on separate computers.

The methods, steps, systems, and system elements described above may be implemented in software, hardware or firmware, or any combination of the three, as part of the computer system described above or as an independent component.

Such methods, steps, systems and system elements, either individually or in combination, may be implemented as a computer program product tangibly embodied as computer-readable signals on a computer-readable medium, for example, a non-volatile recording medium, an integrated circuit memory element, or a combination thereof. For each such method and step, such a computer program product may comprise computer-readable signals tangibly embodied on the computer-readable that define instructions, for example, as part of one or more programs, that, as a result of being executed by a computer, instruct the computer to perform the method or step.

The invention may be more fully understood by reference to the following examples. These examples, however, are merely intended to illustrate the embodiments of the invention and are not to be construed to limit the scope of the invention.

### EXAMPLES

Materials and Methods

Isolation of Normal Human Monocytes and Leukocytes: Ficoll Separation of Monocytes and Neutrophils from Human Leukopacks Monocyte Isolation

35 ml of leukopack suspension (provided by the Dana Farber Cancer Institute blood bank, Boston, Mass.) was placed in a 50 ml conical tube, underlayed with 15 ml of Ficoll-Paque (Pharmacia, Piscataway, N.J.), and spun at 1800 rpm for 25 minutes. The mononuclear layer was collected into two 50 ml tubes and washed with 1× sterile phosphate buffered saline (PBS) (1200 rpm for 10 minutes). The red blood cell/white blood cell upper layer was saved for further processing. Twice, the mononuclear samples were resuspended in 5 ml of EDTA serum (500 ul of 105 mM EDTA with pH=7.4 in 10 ml human serum (Sigma, St. Louis, Mo.) for a final of 5 mM EDTA), incubated at 37C. for 10 minutes, and spun at 1200 rpm. The pellets were washed twice with sterile PBS (spun at 1200 rpm for 10 minutes) and then pooled and resuspended with 50 ml of sterile PBS. Cells were counted with a hemocytometer (usually 33% are monocytes). Cells were spun and then resuspended in RPMI 1640 (Cellgro, Herndon, Va.) with 10% human serum and 1% penicillin-streptomycin (Celigro, Herndon, Va.) at 2-5×10⁶ monocytes/ml. 2 ml of cells were plated with 8 ml of RPMI/10% serum in Falcon Petri dishes and incubated for 2 hours at 37C. in a CO2 incubator. Non-adherent cells were then aspirated and the adherent layer washed 3 times with sterile PBS. 10 ml of RPMI/10% serum was added and then the cells reincubated at 37C. The media was changed every 3 days. The monocyte layer became confluent at 6 days. The presence of a predominance of monocytes was confirmed by morphology with May Grunwald Giemsa staining, after gently scraping the cells off the plate, and by the presence of CD14 by flow cytometry. At 6 days, the plates were washed 3 times with sterile PBS, TRIzol reagent (GIBCO/BRL, Rockville, Md.) was added, and the samples stored at −20C.

Neutrophil Isolation

Wintrobe tubes were filled with the red blood cell/white blood cell layer from the above Ficoll separation and spun at 2000 rpm for 10 minutes. The plasma was eliminated and the buffy coat recovered. The presence of an overwhelming neutrophil predominance in the sample was confirmed by morphology with May-Grunwald Giemsa staining. TRIzol was added and the samples stored at −20C.

Cell Culture

HL60 cells, provided by American Type Cell Culture (Manassas, Va.), were grown in RPMI medium 1640 with 10% fetal bovine serum (Sigma, St. Louis, Mo.) and 1% penicillin-streptomycin. For design of the model, neutrophil differentiation was stimulated with 1 uM ATRA (Sigma, St. Louis, Mo.) for 0, 24, 48, 72, and 120 hours and with 1.25% dimethyl sulfoxide (DMSO) (American Bioanalytical, Natick, Mass.) for 72 hours. Monocyte/macrophage differentiation was induced with 10 nM phorbol 12-myristate 13-acetate (PMA) (Sigma, St. Louis, Mo.) for 0, 4, 12, and 24, and 120 hours and with Vitamin D3 (1 alpha 25-dihydroxy) (Calbiochem, San Diego, Calif.) 2.5 uM for 72 hours. Differentiation was confirmed by morphological changes by light microscopy for monocyte/macrophage differentiation and by May Grunwald Giemsa stain for neutrophil differentiation.

In the actual chemical library screen, HL60 cells were grown at 0.45×10⁶/ml in 40 ul of RPMI 1640 media with 10% fetal bovine serum and 1% penicillin-streptomycin in 384 well 120 ul Falcon cell culture plates. Sixteen control wells per 384 well plate consisted of media only (negative control), undifferentiated cells, 1 uM ATRA (neutrophil differentiation), and 10 nM PMA (Monocyte/macrophage differentiation). Chemicals from an approximately 1700 compound library of known biologically active compounds were added at 40 nl for a final concentration of 4 ug/ml. The cells were incubated at 37C. in a 5% CO2 incubator for 3 days.

Expression Analysis

RNA prepared using TRIzol reagent was used to generate first strand cDNA by using a T7-linked oligo (dT) primer. After second strand synthesis, in vitro transcription (with T7 MEGASCRIPT Kit (Ambion, Austin, Tex.)) was performed using biotinylated UTP and CTP (ENZO Diagnostics, New York, N.Y.). 40 ug of biotinylated RNA was fragmented and hybridized overnight to Affymetrix HuFL arrays containing probes for 6800 genes and Affymetrix U95A v2 arrays containing probes for 12,600 genes. After washing, the arrays were stained with streptavidin-phycoerythrein (Molecular Probes, Eugene, Oreg.) and scanned on a Hewlett Packard scanner. Fluorescent intensities were analyzed with GENECHIP software (Affymetrix, Santa Clara, Calif.). For the HuFL arrays, a threshold of 100 was assigned to any gene with a calculated expression value of less than 10 and a threshold of 20,000 was assigned to any gene with an expression level over 20,000. For the U95A v2 arrays a threshold of 10 was assigned to any gene with a calculated expression value of less than 10 and a threshold of 16,000 was assigned to any gene with an expression level over 16,000. Nearest neighbor analysis was used to identify genes with a near binary expression pattern in patient AML cells versus normal human monocytes and neutrophils. These signatures were confirmed to be discriminatory in an HL60 cell line model of hematopoietic differentiation to a monocyte/macrophage phenotype with PMA and to a neutrophil phenotype with ATRA.

High Throughput RNA Extraction and Reverse Transcription

Cells were grown in 384 well format as described above. We created a high throughput protocol for RNA extraction and RT-PCR by modifying the Express Direct mRNA Capture and RT System for RT-PCR by Pierce (Rockford, Ill.). All Pierce reagents were used for the following steps. 45 ul of lysis buffer mixture containing 1× Lysis I Reagent (a hypotonic buffer), 2 mM DTT, 500 units/ml RNase Inhibitor, and 2.4 ul of Lysis II Reagent (a detergent buffer) were added to each well, mixed 5 times, and kept on ice for 30-40 minutes. 6 ul of a 2.5× binding buffer were added per well to a 384 well plate custom coated with oligodT provided by Pierce. Other methods for high throughput mRNA extraction exist and can be used in the methoods of the invention. For instance another method is oligo dT coated magnetic beads (for example Kingfisher by Labsystems, Inc.). 16 ul of the cellular lysate was added per well to the oligodT coated plate. The plate was placed on a plate shaker for 15-20 minutes at a setting of 4 to allow for mRNA binding. The solution was then spun out of the plate into a Super Rag at 700 rpm for 1 minute. The wells were washed twice with the Low Salt Wash Buffer (20 ul/well/wash). Buffer was removed between washes by spinning the buffer out into a Super Rag at 700 rpm for 1 minute. 20 ul/well of 1× first strand cDNA mix was added to the plate with bound mRNA (1 ml of cDNA mix contained 333 ul of 3×cDNA mix, 60 ul of 0.1M DTT, and 607 ul of DEPC treated water). The plate was placed at 37C. for 1.5 hours.

Multiplexed PCR

Primer 3 software was used to design PCR primers (http://www-genome.wi.mit.edu/cgi-bin/primer/primer3_www.cgi). To eliminate the possibility of amplification of contaminating genomic DNA, PCR primers were designed to span a large intron. Primers contained 19 to 22 sequence specific nucleotide and a 9-20 nucleotides tag of nonspecific sequence (GIBCO BRL, Rockville, Md.). The addition of a tag prevented these PCR primers from interfering with the assessment of SBE/MALDI-TOF data. Amplicons were 118 to 384 nucleotides in size. 20 ul of PCR mix per well were added. 1× PCR buffer (Perkin Elmer, Wellesley, Mass.), 5 mM MgCl₂ (Perkin Elmer), 2.5 mM dNTPs, 0.05 uM each primer (GIBCO BRL, Rockville, Md.), and 0.15 units/rx Taq (AmpliTaq Gold, Perkin Elmer) were used. In an MJ 384 well thermocycler, samples were incubated at 92C. for 9 minutes and then 30 cycles at 92C. for 30 s, 65C. for 30 s, and 72C. for 1 minute were performed. A final extension at 72C. for 5 minutes completed the PCR.

| | |
|---|---|
| PCR Primer Name | Primer Sequence |

| | |
|---|---|
| Glyceraldehyde 3- | |
| dehydrogenase | |
| (M33197) | |
| GAPDH FT7 | TAATACGACTCACTATAGGGAGAAGCCACATCGCTCAGACAC (SEQ ID NO.:1) |
| GAPDH RT3 | AATTAACCCTCACTAAAGGGAGACTCCATGGTGGTGAAGACG (SEQ ID NO.:2) |
| Interleukin 1 | |
| Receptor | |
| Antagonist | |
| (X53296) | |
| IL1Rn FT7 | TAATACGACTCACTATAGGGAGACTGGGATGVFAACCAGAAGACC (SEQ ID NO.:3) |
| IL1Rn RT3 | AATTAACCCTCACTAAAGGGAGAAGCTGGAGTCTGGTCTCATCA (SEQ ID NO.:4) |
| Secreted | |
| Phosphoprotein 1 | |
| (U20758) | |
| SPP1 FT7 | TAATACGACTCACTATAGGGAGATTTTGCCTCCTAGGCATCAC (SEQ ID NO.:5) |
| SPP1 RT3 | AATTAACCCTCACTAAAGGGAGATGTGGGGCTAGGAGATTCTG (SEQ ID NO.:6) |
| 47 kD Chronic | |
| granulomatous | |
| disease protein | |
| (M55067) | |
| NCF1 FT7 | AGCGGATAACAGTCCTGACGAGACGGPAGA (SEQ ID NO.:7) |
| NCF1 RT3 | AGCGGATMCCGTCCAGGAGCTTGTGAATTA (SEQ ID NO.:8) |
| Orosomucoid | |
| (X02544) | |
| ORM FT7 | TAGGTTGACAAGCTCTCGACTGCTTGTGC (SEQ ID NO.:9) |
| ORM RT3 | TAGGTTGACCTCTCCT1CTCGIGCTGCTT (SEQ ID NO.:10) |

Single Base Extension (SBE) with Matrix Assisted Laser Desorption (MALDI) Time of Flight (TOF) Mass Spectrometry (Sequenom Detection)

5 ul of PCR product were transferred to a Marsh 384 well plate. 2 ul of a mixture containing 1.7 ul of 0.5× Thermosequenase buffer and 0.3 ul of Shrimp alkaline phosphatase (SAP) (Sequenom, San Diego, Calif.) (1 unit/ul) were added to each well and the plate placed in an MJ Thermocycler at 34C. for 20 minutes and then 85C. for 5 minutes to inactivate any remaining free dNTPs. 2 ul of the SBE mix (1× Thermosequenase buffer, 2.7 uM of each primer, 0.2 mM of each ddNTP (Sequenom), and 0.58 units/rx of Thermosequenase (Sequenom) were then added. The plate was placed in an MJ Thermocycler (after incubation at 92C. for 2 minutes, then 40 cycles at 92C. for 20 seconds and 50C. for 30 seconds were performed). The SBE product was then treated with 16 ul of resin (Sequenom) and then spotted (Spectropoint by Sequenom) and then detected (Biflex mass spectrometer by Bruker, Billerica, Mass.).

| | | |
|---|---|---|
| SBE Probe | Probe Sequence | Terminator |

| | | |
|---|---|---|
| GAPDH_T | ATGGGGAAGGTGAAGG (SEQ ID NO.:11) | T |
| IL1Rn_T | CATTGAGCCTCATGCTC (SEQ ID NO.:12) | T |
| SPP1_G | TACAACAAATACCCAGATGCT (SEQ ID NO.:13) | G |
| NCF1_G | AAGGCCTACACTGCTGTG (SEQ ID NO.:14) | G |
| ORM_C | CCCAGGTCAGATGTCATGTA (SEQ ID NO.:15) | C |

Custom Spotted Microarray Detection of Multiplex PCR Amplicons

Another way of detecting the relative amounts of DNA (and gene expression profile) was by spotted microarray method. The presence or absence of these unique genes after exposure of the given cell line to a chemical compound was determined by spotting unpurified PCR amplicon from the cDNA preps from each chemical query onto a microarray. The PCR amplicon was mixed with a spotting buffer (e.g. 5.5 M NaSCN) and then spotted via microarray technology quill pin onto a glass surface. Other spotting means may also be used such as ring/pin tool, solid pins or piezo electric deposition. The glass surface was derivatized with aminosilane (although other slide coatings are possible substitutes such as polylysine or aldehyde silane). The spotted unpurified PCR amplicon was then immobilized either by baking or UV crosslinking. The spotted immobilized PCR amplicons were then boiled in sterile water for 2 minutes to denature the PCR duplex into hybridizable ssDNA.

The genes specific for each phenotype were detected by a two step “fluorescence signal amplification” stain procedure developed at the Whitehead Institute/MIT Center for Genome Research (Cambridge, Mass.). Each PCR amplicon immobilized to the glass solid support was detected by the capture of a fluorescently-labeled DNA dendrimer stain (3DNA Genisphere Inc., Montvale, N.J.). The DNA dendrimer was a complex of DNA duplexes having end- labeled fluorescent dyes attached to the dendrimer.

Two methods of DNA capture were employed wherein the 3DNA dendrimers were custom made to contain recognition sequences directly taken from the genes of interest (referred to as direct gene dendrimer method) or by using a bridge oligo called a “bipartite probe”. In the case of the bipartite probe, a DNA sequence was used wherein one half of the oligo hybridized to the immobilized PCR amplicon of interest while the second half of the bipartite probe hybridized and captured the specific 3DNA dendrimer. The direct gene dendrimer method involves overnight incubation of DNA on a surface with a dendrimer having 350 dyes attached and a capture sequence which is complementary to the DNA on the surface. Empirically we have found that having the gene sequence directly attached to the dendrimer yielded both higher specificity and sensitivity for multiplex applications over the bipartite dendrimer capture method. Routinely four bipartite probes in the bipartite methods were used to capture the specific dye-labeled dendrimer. The system was optimized to evaluate 3 genes simultaneously on a microarray scanner using ALEXA, CY5 and Cy3 dendrimers.

The first stain step was done by hybridizing 6 uM bipartite probes (4 per gene) to the microarray. The slide was incubated at 45° C. for 45 minutes with coverslip in a humidifying chamber. The slides were washed and then hybridized with the appropriate amount of 3DNA dendrimer. The slides were again incubated at 45° C. for 45 minutes. The slides were washed and then dried by centrifugation and scanned in three colors for each of the three genes.

### Example 1

Defining the Marker Gene Set

Acute Myeloid Leukemia (AML) represents a form of cancer for which improved treatment in therapy is greatly sought. For more than 20 years, researchers have used a cell line (HL60 cell line) which closely mimics the behavior of AML and which is easily maintained in cell culture. The HL60 cells, upon exposure to all trans retinoic acid (ATRA), differentiated into neutrophils. In addition, HL60 cells exposed to Phorbol 12-myristate 13-acetate (PMA) differentiated into monocyte/macrophage. Experimentally, the gene expression differences between HL60 cell and either PMA or ATRA differentiated cells were obtained using high density DNA microarrays (Affymetrix). The different gene expression signatures were evaluated for “in vivo authenticity” by confirming the same gene expression differences in native human neutrophils and native human monocytes compared to patient AML cells. A time course was also done to define the optimum time required for each gene expression signature induction. This added level of “in vivo” specificity was important for yielding “Hits” from the primary screen, which were capable of in vivo activity. From this analysis, sets of genes were obtained which faithfully conveyed the phenotypic change between undifferentiated HL60 cells and monocyte and neutrophil signatures. The two genes that robustly defined the monocyte phenotype were (1) Interleukin 1 receptor antagonist (IL1RN) (X53296 ) and (2) Secreted phosphoprotein 1 (SPP1) (U20758). The neutrophil state was well defined by use of (1) Orosomucoid (ORM1) (X02544) and (2) 47 kD autosomal chronic granulomatous disease protein gene (NCF1) (M55067). These genes, as well as the HL60 cell line, were then tested for their ability to be used in a high throughput process to identify novel agents that induced HL60 differentiation.

### Example 2

High Throughput Cell Culture and Drug Screen

The HL60 cell line was cultured in high throughput plate format (96, 384 1536). The HL60 cell line could also be cultured by some other means using standard robotic dispensing instrumentation. The cells were exposed to small molecules from a chemical library and incubated over the optimum time required to observe the gene expression signature.

### Example 3

High Throughput Capture and Amplification of mRNA Transcripts

The mRNA from the “interrogated” cells were extracted in high throughput format. One such format available from Pierce employed plastic 384 well plates coated with covalently attached oligo dT DNA. The interrogated cells were passed through a two step lysis procedure. The lysis solution containing mRNA was applied to the 384 well oligo dT plates. An incubation time was allowed to enable the poly A tails on the mRNA transcripts to hybridize to the solid phase. The plate underwent stringency washes and then reverse transcription by a M-MuLV reverse transcriptase reaction. The process of reverse transcription converted the mRNA into DNA and allowed the transcript to be retained covalently on the solid phase (plastic walls of plate). The converted DNA transcripts (now called cDNA) were then amplified by PCR. Primers specific for each marker gene were added to each well and then amplified by standard PCR.

### Example 4

High Throughput Detection of Amplified Transcripts

The PCR products were then read out on either spotted microarray or by Single base extension (SBE) using a Sequenom Mass spectrometer. SBE by Sequenom involved adding primers specific to the internal region of each amplified PCR fragment. The SBE reaction by Sequenom was readable in multiplex format (7 plex reaction readouts). The SBE reaction mixture was spotted in 384 well format onto a MALDI matrix coated disk and detected by mass spectrometry. The signal to noise ratio was determined relative to the good housekeeping genes (see data analysis).

The custom spotted array was performed directly on the PCR amplicons. The PCR fragments of each marker gene were spotted in an array format using a microarraying device. The spotted PCR fragments were then UV crosslinked and then boiled to open up the dsDNA PCR amplicons. The spotted array was then stained using a fluorescent amplifying stain such as 3DNA dendrimer staining or by other detection method such Quantum dots, Tyramide assay (NEN) or rolling circle DNA amplification (RCA, Molecular Staging). The scanned image was converted into a tif file and data were extracted by a standard microarray extraction program (Arrayvision, Quantarray, Axon).

Results

Control genes were tested across twelve 384 well plates processed by the methods as described in the methods section. Each plate contained at least 16 samples of each phenotypic control. Each plate contained representative examples of three phenotypes namely undifferentiated HL60 cells (“AML”), cells which have been chemically differentiated by phorbol ester (PMA) causing the monocyte phenotype, and cells which have been exposed to all Trans retenoic acid (ATRA) which will induce a neutrophil phenotype. The three phenotypes are defined as either undifferentiated (AML), neutrophil as ATRA (the inducing chemical), and monocyte phenotype by PMA (the inducing chemical the phorbol ester). The genes for distinguishing the monocyte phenotype were IL1RN, SPP1 and GAPDH. The genes for distinguishing the neutrophil phenotypic signature were NCF1, Orosomucoid and GAPDH. The intensities for each gene were measured either by Sequenom mass spectrometer or by spotted microarray with fluorescent dendrimer bipartite staining or direct gene dendrimer methods.

The phenotypic signature was derived by a ratio of the up-regulated genes to the “good housekeeping” gene (GAPDH). Hence, the monocyte phenotype was represented by ratio of IL1RN/GAPDH and SPP1/GAPDH. The intensity ratios for NCF1/GAPDH and ORO/GAPDH represented the neutrophil signature. The raw ratios from each detection method were filtered by taking the average intensity for the negative control wells plus one standard deviation. The negative control wells were wells, which contained only PCR reaction mix but no cellular material such as mRNA. An internal filter was applied in order to prevent evaluation of failed spotting and or detection by either method. The mass spectrometer data was collected/processed individually on each of the twelve plates, while the microarray was taken from a single slide printing of all twelve plates.

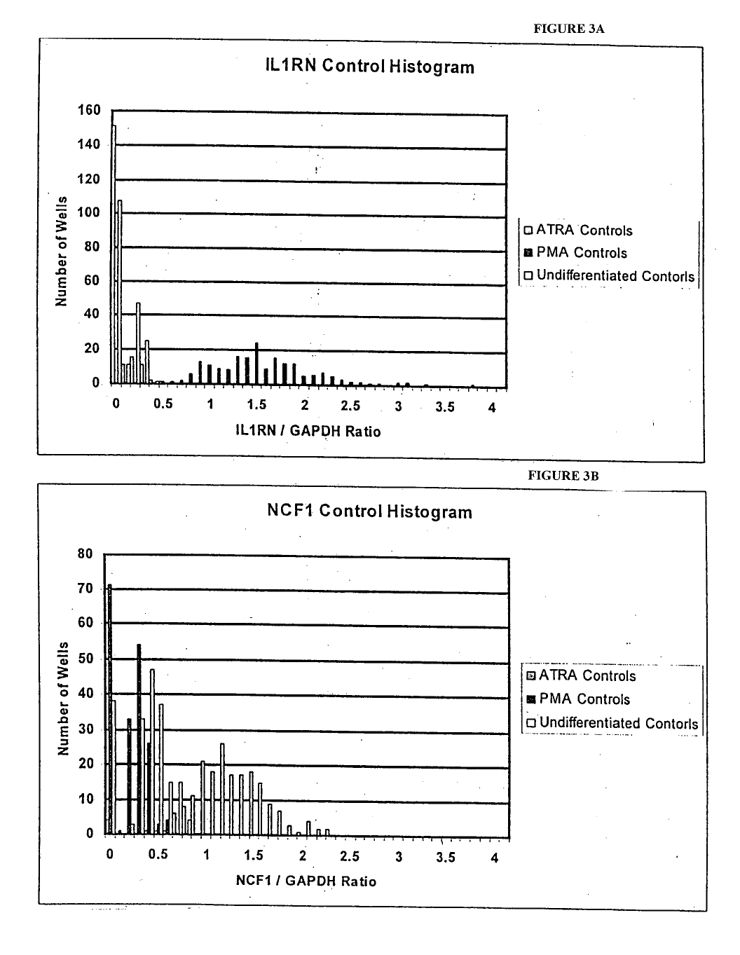

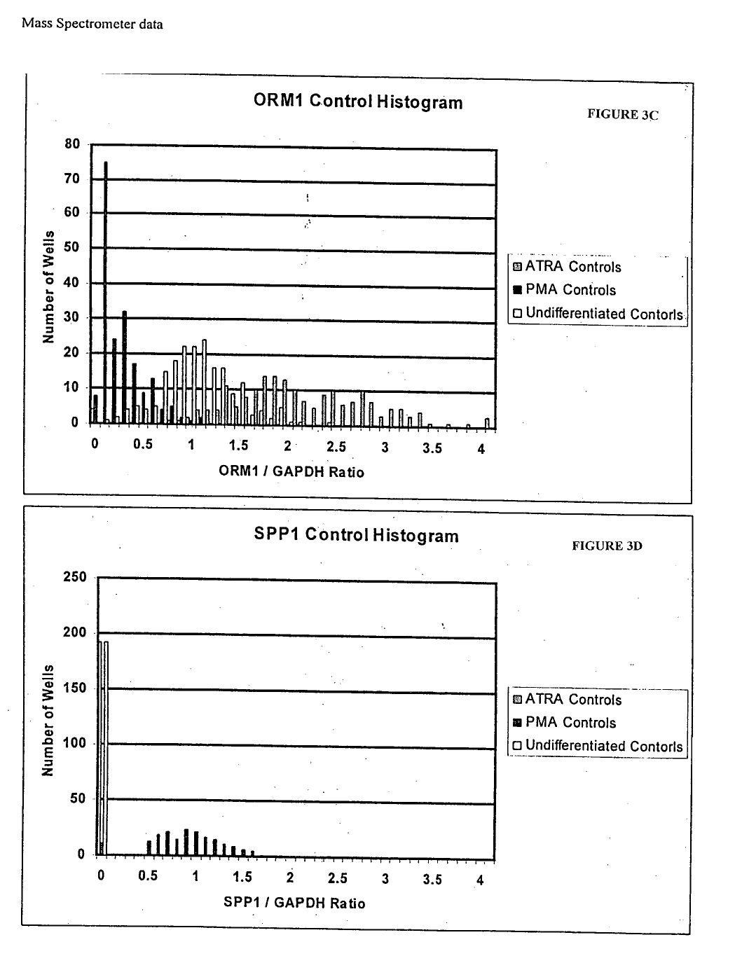

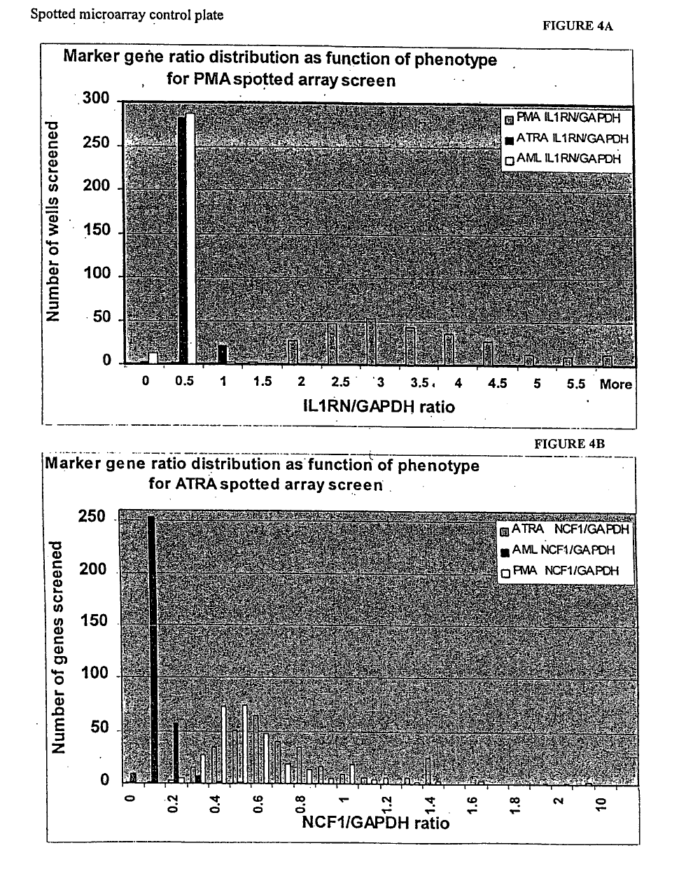

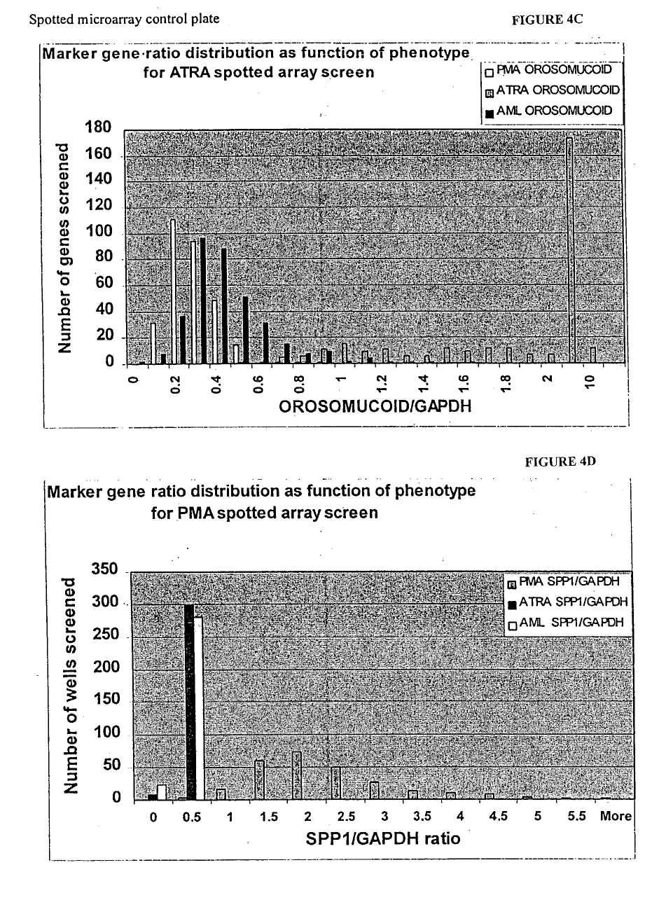

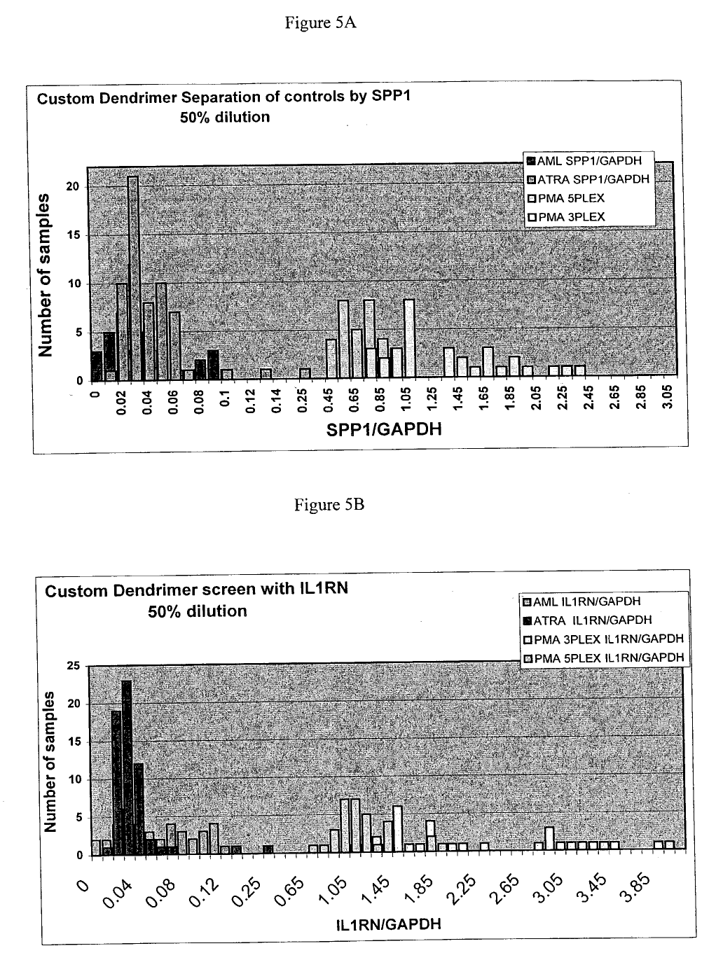

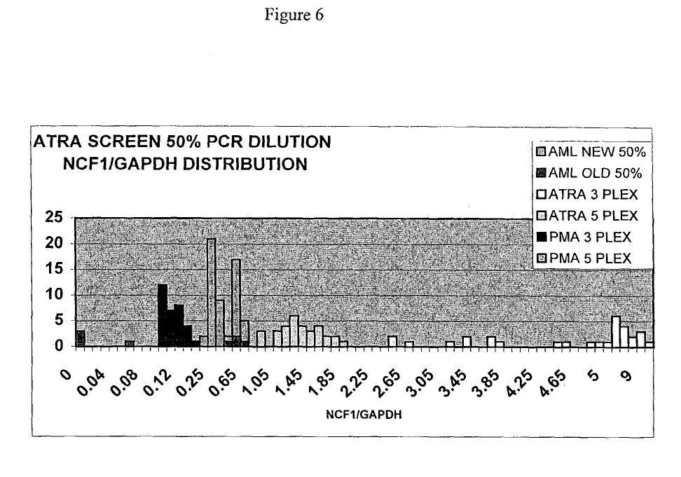

The ratios of each known control phenotype were then plotted as a histogram to display the distribution of the gene intensity ratios. FIGS. 3-4 (bipartite probe) and 5-6 (direct gene dendrimer) depict the number of genes having a particular ratio of gene intensity. As can be seen from all graphs there is a distribution of ratios present in all three phenotypes that were observed in both detection methods (mass spectrometer (FIGS. 3A-3D and 5A-6) and spotted array (FIGS. 4A-4D)). An important parameter of each detection method is the ability of each detection method to adequately separate the AML or undifferentiated HL60 cell line ratio signature away from either the monocyte or neutrophil ratio signature. The mass spectrometer ratio data for IL1RN/GAPDH (FIGS. 3 and 5B) and SPP1/GAPDH (FIGS. 6 and 5A) revealed a very clean separation between the monocyte signature of the PMA induced HL60 cells and the HL60 uninduced cells. A slight overlap between the ratios for the HL60 cells and neutrophil (ATRA induced cells) phenotype was observed, yet, application of the correct threshold easily defines a separation of roughly 90%. Similarly, the spotted microarray data also gave strong separation between the control states of undifferentiated cells and the neutrophil and monocyte phenotype.

The values demonstrated in FIG. 5 are presented in the Table below.

| | | | | | IL1RN | SPP1 |
|---|---|---|---|---|---|---|
| Highest stringency filter of 50% dilution | | | | | fold | fold |
| | Ave | | Ave | | increase | increase |
| | IL1RN/ | | SPP1/ | | over | over |
| | GAPDH | Sdev | GAPDH | Sdev | AML | AML |
| AML | 0.02 | 0.01 | 0.02 | 0.01 | | |
| ATRA 3 plex | 0.02 | 0.01 | 0.03 | 0.01 | | |
| ATRA 5 plex | 0.02 | 0.01 | 0.02 | 0.01 | | |
| PMA 3 plex | 2.04 | 0.78 | 1.17 | 0.36 | 84.4 | 55.1 |
| PMA 5 plex | 0.98 | 0.24 | 0.55 | 0.13 | 40.5 | 25.9 |

As shown in the last two columns of the Table, IL1RN was increased 40-84 fold in PMA treated cells with respect to AML and SSP1 was increased 25-55 fold over AML.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. The advantages and objects of the invention are not necessarily encompassed by each embodiment of the invention. All references, patents and patent publications that are recited in this application are incorporated in their entirety herein by reference.

We claim:

1. A method of determining a gene expression profile for a cellular phenotype comprising:
establishing two or more sets of gene expression profiles;
defining a set of marker genes that defines the differences between the two or more sets of gene expression profiles; and
recording the set of marker genes in a database that defines the cellular phenotype.

2. A method of screening a cell population comprising:
defining a set of marker genes that represents a cellular phenotype;
amplifying the set of marker genes from the cell population;
determining the expression of the marker genes present in the cell population; and
scoring the expression of the marker genes to screen the cell population for the cellular phenotype.

3. The method of claim 2, wherein the cell population is a cultured cell line.

4. The method of claim 2, wherein the cell population is an in vivo cell population.

5. The method of claim 2, wherein the marker genes are scored relative to the expression of a control gene.

6. The method of claim 2, wherein the marker genes are scored relative to each other.

7. The method of claim 2, wherein the marker genes are scored on a binary basis.

8. The method of claim 2, wherein the control gene is GAPDH.

9. The method of claim 2, wherein the cellular phenotype is of a cancer cell.

10. The method of claim 2, wherein the cellular phenotype is of a metastatic cancer cell.

11. The method of claim 2, wherein the cellular phenotype is of a cell resistant to radiation.

12. The method of claim 2, wherein the cellular phenotype is of a cell resistant to chemotherapy.

13. The method of claim 2, wherein the cellular phenotype is of a cancer cell that releases angiogenic factors.

14. The method of claim 2, wherein the cellular phenotype is of a cell with a positive drug response.

15. The method of claim 2, wherein the set of marker genes defines a set of phenotypic markers.

16. The method of claim 2, wherein the set of marker genes defines a set of therapeutic markers.

17. The method of claim 2, wherein the set of marker genes defines a set of diagnostic markers.

18. The method of claim 2, wherein the cell population is a population of cells from human peripheral blood.

19. The method of claim 2, wherein the set of marker genes defines 1 or more novel genes.

20. The method of claim 2, wherein the set of marker genes represents a biological pathway.

21. The method of claim 2, wherein the set of marker genes defines a transcriptome.

22. The method of claim 2, wherein the cell population is screened in response to a chemical compound.

23. The method of claim 22, wherein the chemical compound is selected from the group consisting of small molecule libraries, FDA approved drugs and synthetic chemical libraries.

24. The method of claim 2, wherein 1 or more of the marker genes is selected from the group consisting of IL1RN and SPP1.

25. The method of claim 2, wherein 1 or more of the marker genes is selected from the group consisting of ORM1 and NCF1.

26. The method of claim 2, wherein the expression of the marker gene is detected using a bipartite probe.

27. The method of claim 2, wherein the expression of the marker gene is detected using direct gene dendrimer methods.

28. The method of claim 2, further comprising:
defining one or more metagenes in response to one or more drugs.

29. The method of claim 28, wherein the drug is an anti-cancer drug.

30. A method for identifying an active compound, comprising:
contacting cells with a plurality of chemical compounds,
amplifying a set of marker genes from the cells to determine the expression of marker genes present in the cells, and
scoring the expression of the marker genes to identify a cellular phenotype, the presence of a specific cellular phenotype being indicative of an active compound.

31. The method of claim 30, wherein the plurality of chemical compounds is a set of compounds selected from the group consisting of small molecule libraries, FDA approved drugs, synthetic chemical libraries, phage display libraries, dosage libraries.

32. The method of claim 30, wherein the set of marker genes is a metagene.

33. The method of claim 30, wherein the active compound is an anti-cancer drug.

34. The method of claim 33, wherein the cellular phenotype is a tumorigenic status of the cell.

35. The method of claim 33, wherein the cellular phenotype is a metastatic status of the cell.

36. The method of claim 30, wherein the set of marker genes is a cancer versus non-cancer marker gene set.

37. The method of claim 30, wherein the set of marker genes is a metastatic versus non-metastatic marker gene set.

38. The method of claim 30, wherein the set of marker genes is a radiation resistant versus radiation sensitive marker gene set.

39. The method of claim 30, wherein the set of marker genes is a chemotherapy resistant versus chemotherapy sensitive marker gene set.

40. The method of claim 30, wherein the active compound is a cellular differentiation factor.

41. The method of claim 40, wherein the cellular phenotype is a cellular differentiation status.

42. The method of claim 30, wherein the expression of the marker genes is determined by custom reverse microarray analysis.

43. The method of claim 30, wherein the expression of the marker genes is determined by mass spectrometry.

44. A method for identifying a cellular phenotype, comprising:
identifying the expression of metagenes in a cell to identify a cellular phenotype of the cell.

45. The method of claim 44, wherein the expression of metagenes is identified by amplifying signature genes characteristic of the metagenes from the cells.

46. The method of claim 45, wherein the cellular phenotype is identified by scoring the expression of the metagenes on a binary basis.

47. The method of claim 45, wherein the cellular phenotype is a cellular differentiation status.

48. The method of claim 45, wherein the cellular phenotype is a tumorigenic status of the cell.

49. A method for identifying a function of a gene, comprising:
contacting cells with a diverse array of chemical compounds,
amplifying a set of marker genes characteristic of a transcriptome from the cells to determine the expression of the marker genes present in the cells,
identifying a gene with an unknown function based on the expression of the marker genes, and
correlating an activity of one or more chemical compounds from the diverse array to the gene with unknown function to identify a function for the gene.

50. A method for identifying an active compound, comprising:
contacting cells with a plurality of chemical compounds,
screening proteins isolated from the cells to determine expression of a set of marker proteins, and
scoring the expression of the marker proteins to identify a cellular phenotype, the presence of a specific cellular phenotype being indicative of an active compound.

51. A method for identifying changes in cellular proliferation, comprising:
contacting cells with a plurality of chemical compounds,
amplifying at least one control gene from the cells,
scoring the level of expression of the control gene to determine a relative amount of cellular proliferation with respect to a level of expression of the control gene in a similar cell.

52. A database representing a library of phenotypic states of cells, the database tangibly embodied on a computer-readable medium and comprising:
one or more phenotype data structures, each phenotype data structure representing a phenotypic state and including at least one marker data unit representing a marker and specifying a difference in an expression level of the marker for a cell having the phenotypic state and an expression level of the marker for a biological cell not having the phenotypic state.

53. A data structure representing a phenotypic state of a cell, the data structure tangibly embodied on a computer-readable medium and comprising:
at least one marker data unit representing a marker and specifying a difference in an expression level of the marker for a cell having the phenotypic state and an expression level of the marker for a biological cell not having the phenotypic state,
wherein the marker data unit was generated using reverse gene expression analysis.

54. A method of determining whether a chemical compound applied to undifferentiated cells produces differentiated cells exhibiting a phenotype, the method comprising acts of:
(A) receiving expression levels of nucleic acids of a sample from an array of samples, the sample produced from introducing at least one of the undifferentiated cells to a chemical well containing the chemical compound;
(B) determining whether the chemical well from which the sample resulted is a dead chemical well by determining whether the resulting expression level of a housekeeping nucleic acid of the spot sample reaches a threshold expression level value;
(C) if the expression level of the housekeeping gene reaches the threshold value, normalizing an expression level of at least a first nucleic acid that is a marker for the phenotype;
(D) determining whether the normalized expression level reaches a threshold level indicative of the chemical compound producing differentiated cells from the undifferentiated cells.

55. The method of claim H1, wherein act (A) comprises:
for each receiving a first signal representing the expression level of a housekeeping gene.

56. The method of claim H1, wherein at least part of the method is implemented using a computer.
